(19) United States
(12) Patent Application Publication
(10) Pub. No.: US 2001/0039286 A1
(43) Pub. Date: Nov. 8, 2001

(54) 2-aryl indole derivatives and their use as therapeutic agents

(76) Inventors: Kevin Dinnell, Much Hadham (GB); Jason Matthew Elliott, Felsted (GB); Gregory John Hollingworth, Brentwood (GB); Mark Peter Ridgill, Watton-at-Stone (GB); Duncan Edward Shaw, Bishops Stortford (GB)

Correspondence Address: MERCK AND CO INC, P O BOX 2000, RAHWAY, NJ, 070650907

(21) Appl. No.: 09/782,422

(22) Filed: Feb. 13, 2001

(30) Foreign Application Priority Data
Feb. 14, 2000 (GB) 0003397.7

(51) Int. Cl. A61K 31/454; A61K 31/4439; C07D 401/00

(52) U.S. Cl. 514/320; 514/337; 546/201; 546/278.1

(57) ABSTRACT

The present invention relates compounds of the formula (I):

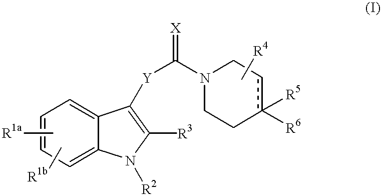

wherein

R^{1a}, R^{1b}; and R² represent a variety of substituents;

R³ represents an optionally substituted phenyl, biphenyl or naphthyl or heteroaryl group;

R⁴ represents hydrogen, C₁₋₆alkyl, carbonyl (=O), (CH₂)ₚphenyl or a

C₁₋₂alkylene bridge across the piperidine ring;

R⁵ and R⁶ each independently represent a variety of substituents; or

R⁵ and R⁶ together are linked so as to form an optionally substituted 5-or 6-membered ring;

X represents an oxygen or a sulfur atom, two hydrogen atoms, ═NH or ═N(C₁₋₆alkyl);

Y is a straight or branched C₁₋₄alkylene, C₂₋₄alkenylene or C₂₋₄alkynylene chain;

the dotted line represents an optional double bond;

m is zero or an integer from 1 to 4; n is an integer from 1 to 4; and p is an integer from 1 to 4;

or a pharmaceutically acceptable salt thereof.

The compounds are of particular use in the treatment or prevention of depression, anxiety, pain, inflammation, migaine, emesis or postherpetic neuralgia.

This invention relates to indole derivatives and their use as tachykinin antagonists, and in particular as neurokinin-1 receptor antagonists.

We have now found a class of indole derivatives which are potent receptor antagonists of tachykinins, especially of the neurokinin-1 (substance P) receptor.

The present invention accordingly provides the compounds of the formula (I):

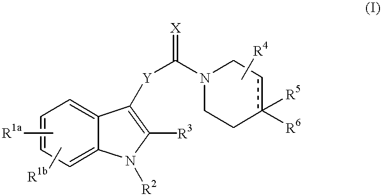

wherein

R^{1a} and R^{1b} each independently represent hydrogen, C₁₋₆alkyl, C₂₋₆alkenyl, C₁₋₆alkoxy, fluoroC₁₋₆alkyl, fluoroC₁₋₆alkoxy, halogen, cyano, NR^{a}R^{b}, SR^{a}, SOR^{a}, SO₂R^{a}, OSO₂R^{a}, NR^{a}COR^{b}, COR^{a}, CO₂R^{a}, CONR^{a}R^{b}, phenyl or heteroaryl, wherein said phenyl or heteroaryl group may be optionally substituted by one, two or three groups independently selected from halogen, C₁₋₆alkyl, C₁₋₆alkoxy, fluoroC₁₋₆alkyl, fluoroC₁₋₆alkoxy, NO₂, cyano, SR^{a}, SOR^{a}, SO₂R^{a}, COR^{a}, CO₂R^{a}, CONR^{a}R^{b}, C₂₋₆alkenyl, C₂₋₆alkynyl, C₁₋₄alkoxyC₁₋₄alkyl or —O(CH₂)₁₋₂O—;

R² represents hydrogen, C₁₋₆alkyl, fluoroC₁₋₆alkyl, (CH₂)ₘCOR^{a}, (CH₂)ₚCO₂R^{a}, (CH₂)ₚOH, (CH₂)ₘCONR^{a}R^{b}, (CH₂)ₘphenyl or SO2C₁₋₆alkyl;

R³ represents phenyl, biphenyl, naphthyl or heteroaryl, wherein said phenyl, biphenyl, naphthyl or heteroaryl group may be optionally substituted by one, two or three groups independently selected from halogen, C₁₋₆alkyl, C₁₋₆alkoxy, fluoroC₁₋₆alkyl, fluoroC₁₋₆alkoxy, NO₂, cyano, SR^{a}, SOR^{a}, SO₂R^{a}, COR^{a}, CO₂R^{a}, CONR^{a}R^{b}, C₂₋₆alkenyl, C₂₋₆alkynyl, C₁₋₄alkoxyC₁₋₄alkyl or —O(CH₂)₁₋₂O—;

R⁴ represents hydrogen, C₁₋₆alkyl, carbonyl (═O), (CH₂)ₚphenyl or a C₁₋₂alkylene bridge across the piperidine ring;

R⁵ and R⁶ each independently represent hydrogen, halogen, C₁₋₆alkyl, C₃₋₇cycloalkyl, C₃₋₇cycloalkylC₁₋₄alkyl, C₂₋₆alkenyl, cyano, phenyl, naphthyl, fluorenyl, heteroaryl, (CH₂)ₚphenyl, (CH₂)ₚheteroaryl, CH(phenyl)₂, CH(C₁₋₆alkyl)(phenyl), C(C₁₋₆alkyl)(phenyl)₂, CO(phenyl), C(OH)(phenyl)₂, C₂₋₄alkenyl(phenyl), (CH₂)ₘNR^{c}R^{d}, (CH₂)ₚCONR^{c}R^{d}, (CH₂)ₚNR^{a}COR^{b}, (CH₂)ₘCOR^{c}, (CH₂)ₘCO₂R^{c} or (CH₂)ₘOH wherein said phenyl, naphthyl, fluorenyl or heteroaryl groups may be optionally substituted by one, two or three groups independently selected from halogen, C₁₋₆alkyl, C₁₋₆alkoxy, fluoroC₁₋₆alkyl, fluoroC₁₋₆alkoxy, NO₂, cyano, SR^{a}, SOR^{a}, SO₂R^{a}, COR^{a}, CO₂R^{a}, CONR^{a}R^{b}, C₂₋₆alkenyl, C₂₋₆alkynyl, C₁₋₄alkoxyC₁₋₄alkyl or —O(CH₂)₁₋₂O—; or

R⁵ and R⁶ together are linked so as to form a 5- or 6-membered ring optionally substituted by ═O, ═S or a C₁₋₄alkyl or hydroxy group, and optionally containing a double bond, which ring may optionally contain in the ring one or two heteroatoms selected from O and S, or groups selected from NR^{c}, SO or SO₂, and to which ring there is either fused or attached a benzene or thiophene ring, which benzene or thiophene ring is optionally substituted by 1, 2 or 3 substituents selected from C₁₋₆alkyl, C₃₋₇cycloalkyl, C₃₋₇cycloalkylC₁₋₄alkyl, phenylC₁₋₄alkyl, trifluoromethyl, cyano, OR^{a}, SR^{a}, SOR^{a}, SO₂R^{a}, NR^{a}R^{b}, NR^{a}CO₂R^{b}, NR^{a}CO₂R^{b}, NR^{a}SO₂R^{b}, CO₂R^{a}, CO₂R^{a} or CONR^{a}R^{b}, wherein the phenyl moiety of a phenylC₁₋₄alkyl group may be substituted by C₁₋₆alkyl, C₁₋₆alkoxy, halogen or trifluoromethyl;

R^{a} and R^{b} each independently represent hydrogen, C₁₋₄alkyl, fluoroC₁₋₄alkyl or phenyl; or

the group —NR^{a}R^{b}may form a 5- or 6-membered ring optionally substituted by ═O, ═S or a C₁₋₄alkyl or hydroxy group, and optionally containing a double bond, which ring may optionally contain in the ring one or two heteroatoms selected from O and S, or groups selected from NR^{c}, SO or SO₂;

R^{c} and R^{d} each independently represent hydrogen, C₁₋₄alkyl, fluoroC₁₋₄alkyl, C₂₋₄alkenyl, COR^{a}, SO₂R^{a}, phenyl or benzyl or R^{c} and R^{d}, together with the nitrogen atom to which they are attached, form a heteroaliphatic ring of 4 to 7 atoms, to which ring there may optionally be fused a benzene ring;

X represents an oxygen atom, a sulfur atom, two hydrogen atoms, ═NH or ═N(C₁₋₆alkyl);

Y is a straight or branched C₁₋₄alkylene chain optionally substituted by halogen, oxo or hydroxy; or

Y represents a straight or branched C₂₋₄alkenylene or C₂₋₄alkynylene chain;

the dotted line represents an optional double bond, with the proviso that when the double bond is present, R⁶ is absent;

m is zero or an integer from 1 to 4;

n is an integer from 1 to 4;

p is an integer from 1 to 4;

or a pharmaceutically acceptable salt thereof.

A preferred group of compounds of formula (I) is that wherein R^{1a} and R^{1b} each independently represent hydrogen, halogen, C₁₋₆alkyl, C₂₋₆alkenyl, fluoroC₁₋₆alkoxy, NR^{a}R^{b}, COR^{a}, CO₂R^{a}, or heteroaryl. Where R^{1a} and R^{1b} are both other than hydrogen, preferably R^{1a} and R^{1b} are the same. Where R^{1a} is other than hydrogen and R^{1b} is hydrogen, R^{1a} is preferably attached to the indole ring at the 5-position.

A particularly preferred group of compounds of formula (I) is that wherein R^{1a} and R^{1b} each independently represent hydrogen, methyl, vinyl, trifluoromethoxy, fluorine, chlorine, bromine, pyrrolidinyl, piperidinyl, morpholino, acetyl, methoxycarbonyl, pyridyl (especially 3-pyridyl) or furyl (especially 2-furyl).

As especially preferred group of compounds of formula (I) is that wherein R^{1a} represents 5-methyl or 5-chloro, and R^{1b} is hydrogen.

A further preferred group of compounds of formula (I) is that wherein R² represents hydrogen, C₁₋₆alkyl, fluoroC₁₋₆alkyl, (CH₂)ₘCOR^{a}, (CH₂)ₚCOR^{a}, (CH₂)ₚOH or (CH₂)ₘphenyl.

A particularly preferred group of compounds of formula (I) is that wherein R² represents C₁₋₃alkyl (especially methyl, ethyl or isopropyl), fluoroC₁₋₃alkyl (especially trifluoromethyl or 2,2,2-trifluoroethyl), COCH₃, CH₂CO₂H, CH₂CO₂CH₃, (CH₂)₁-₂0H (especially CH₂CH₂OH) or benzyl.

An especially preferred group of compounds of formula (I) is that wherein R² is hydrogen or methyl.

Another preferred group of compounds of formula (I) is that wherein R³ represents phenyl, biphenyl, naphthyl (especially 2-naphthyl) or heteroaryl (especially 2- or 3-pyridyl) wherein said phenyl group is optionally substituted by one or two groups selected from halogen, C₁₋₆alkyl, C₁₋₆alkoxy, trifluoroC₁₋₆alkyl, fluoroC₁₋₆alkoxy or C₂₋₆alkenyl.

A particularly preferred class of compounds of formula (I) is that wherein R³ represents phenyl, biphenyl, naphthyl (especially 2-naphthyl) or heteroaryl (especially 2- or 3-pyridyl) wherein said phenyl group is optionally substituted by one or two groups selected from fluorine, chlorine, bromine, C₁₋₄alkyl (especially isopropyl or tertiary butyl), methoxy, trifluoromethyl, trifluoromethoxy or vinyl.

An especially preferred group of compounds of formula (I) is that wherein R³ represents 2-pyridyl, 3-pyridyl or phenyl optionally substituted by one or two groups selected from fluorine, chlorine, bromine, C₁₋₄alkyl (especially isopropyl or tertiary butyl), methoxy, trifluoromethyl, trifluoromethoxy or vinyl.

A most especially preferred class of compounds of formula (I) is that wherein R³ represents phenyl, 4-chlorophenyl, 4-bromophenyl, 4-fluorophenyl, 2-pyridyl or 3-pyridyl.

A further preferred group of compounds of formula (I) is that wherein R⁴ represents hydrogen, methyl, carbonyl, benzyl or a methylene bridge across the 2,5-positions on the piperazine ring.

As especially preferred group of compounds of formula (I) is that wherein R⁴ is hydrogen.

Another preferred group of compounds of formula (I) is that wherein R⁵ represents halogen, C₃₋₇cycloalkyl, C₃₋₇cycloalkylC₁₋₄alkyl, phenyl, heteroaryl, (CH₂)ₚphenyl, (CH₂)ₚheteroaryl, CH(phenyl)₂, CH(C₁₋₆alkyl)(phenyl), C(C₁₋₆alkyl)(phenyl)₂, CO(phenyl), C(OH)(phenyl)₂, or (CH₂)ₚNR^{c}R^{d}, wherein said phenyl or heteroaryl group is optionally substituted by one or two substituents selected from halogen, C₁₋₆alkyl, C₁₋₆alkoxy, fluoroC₁₋₆alkyl, fluoroC₁₋₆alkoxy, NO₂, cyano, SR^{a} or —O(CH₂)₁₋₂O—.

A particularly preferred group of compounds of formula (I) is that wherein R⁵ represents C₅₋₇cycloalkyl (especially cyclohexyl), phenyl, heteroaryl, (CH₂)ₚphenyl (especially wherein p is 1 or 2), CO(p-methoxyphenyl), C(OH)(phenyl)₂, or (CH₂)ₚNR^{c}R^{d} (especially where R^{c} and R^{d} each independently represent hydrogen, C₁₋₄alkyl, C₂₋₄alkenyl, COR^{a} (especially wherein R^{a} is methyl or ethyl), SO₂R^{a} (especially wherein R^{a} is methyl), phenyl or benzyl, or R^{c} and R^{d}, together with the nitrogen atom to which they are attached, form a piperidine ring; and especially wherein p is zero or 1), wherein each of said phenyl or heteroaryl groups may be substituted by one or two groups independently selected from halogen, C₁₋₄alkyl, C₁₋₄alkoxy, fluoroC₁₋₄alkyl, fluoroC₁₋₄alkoxy, NO₂, cyano and SO₂R^{a} (especially wherein R^{a} represents C₁₋₄alkyl), or said phenyl or heteroaryl group may be substituted by the group —O(CH₂)₁₋₂O—. Particularly preferred are compounds in which said phenyl groups are unsubstituted or substituted by one or two substituents independently selected from fluorine, chlorine, methyl, ethyl, methoxy, ethoxy, isopropoxy, trifluoromethoxy, nitro, cyano and thiomethyl, or said phenyl is substituted by —OCH₂—. Also preferred are compounds in which said heteroaryl groups are unsubstituted or are monosubstituted by methyl or trifluoromethyl.

Another preferred class of compounds of formula (I) is that wherein R⁶ represents hydrogen, fluorine, cyano, (CH₂)ₘNR^{c}R^{d}, (CH₂)ₚNR^{a}COR^{b}, (CH₂)ₘCO₂R^{c} or (CH₂)ₘOH, where R^{a}, R^{b}, R^{c} and R^{d} are as previously defined.

A particularly preferred class of compounds of formula (I) is that wherein R⁶ represents hydrogen, cyano, NR^{c}R^{d} (especially wherein R^{c} and R^{d} are both C₁₋₃alkyl, most especially methyl), CH₂NHCOR^{b} (especially wherein R^{b} is C₁₋₃alkyl, most especially methyl), CO₂R^{c} (especially wherein R^{c} is hydrogen or C₁₋₃alkyl, most especially hydrogen or methyl), or (CH₂)ₘOH (especially where m is zero or 1).

Where R⁵ and R⁶ are taken together there is preferably formed a 5-or 6-membered ring optionally substituted by ═O or a hydroxy group, and optionally containing a double bond, which ring optionally contains in the ring an oxygen or sulfur atom or 1 or 2 NH groups, and to which ring is either fused or attached a benzene ring, which benzene ring is optionally substituted by C₁₋₃alkyl or SO₂R^{a}, where R^{a} is as previously defined.

As used herein, NR^{c}R^{d} is preferably NH₂, NHCH₃ or N(CH₃)₂; NR^{a}COR^{b} is preferably NHCOCH₃, N(CH₃)COCH₃ or N(Ph)COCH₃; NR^{a}CO₂R^{b} is preferably NHCO₂CH₃ or N(CH₃)CO₂CH₃; NR^{a}SO₂R^{b} is preferably NHSO₂CH₃, N(CH₃)SO₂CH₃ or N(Ph)SO₂CH₃; and CO₂R^{a} is preferably CO₂H, CO₂CH₃ or CO₂CH₂CH₃.

In particular, when R⁵ and R⁶ are so linked as to form a 5- or 6-membered ring, suitable definitions of the CR⁵R⁶ moiety are selected from:

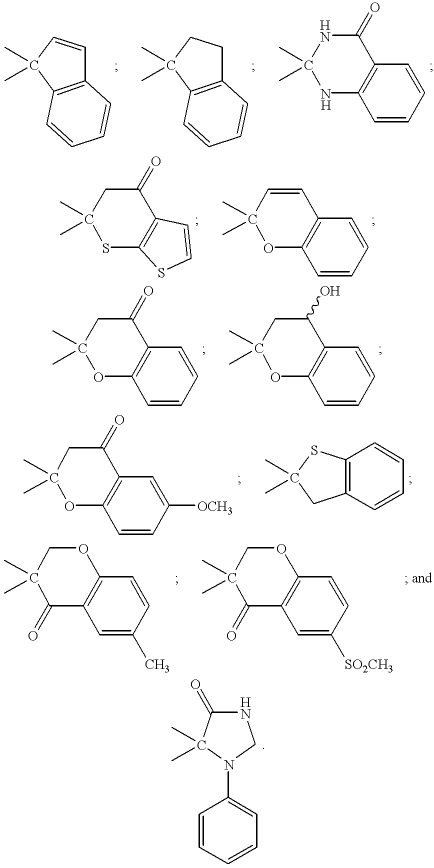

Particularly preferred examples of the CR⁵R⁶ moiety are selected from:

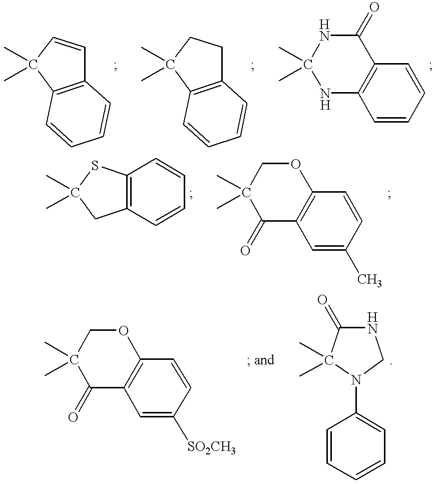

Another preferred group of compounds of formula (I) is that wherein X represents an oxygen atom, two hydrogen atoms, or ═NH. Most especially preferred are compounds wherein X is an oxygen atom.

A further preferred group of compounds of formula (I) is that wherein Y is —CH₂CH₂—, —CH₂CH(CH3)—, —CH═CH— or —C≡C—, and most especially —CH₂CH₂—.

Another preferred group of compounds of formula (I) is that wherein X is two hydrogen atoms and Y is —CH₂CH₂—, —CH₂C(O)—, —CH₂CHOH— or —CH₂CHF—.

When any variable occurs more than one time in formula (I) or in any substituent, its definition on each occurrence is independent of its definition at every other occurrence.

As used herein, the term “alkyl” or “alkoxy” as a group or part of a group means that the group is straight or branched. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy.

As used herein, the terms “fluoroC₁₋₆alkyl” and “fluoroC₁₋₆alkoxy” means a C₁₋₆alkyl or C₁₋₆alkoxy group in which one or more (in particular, 1 to 3) hydrogen atoms have been replaced by fluorine atoms. Similarly, the term “fluoroC₁₋₄alkyl” means a C₁₋₄alkyl group in which one or more (in particular 1 to 3) hydrogen atoms have been replaced by fluorine atoms. Particularly preferred are fluoroC₁₋₃alkyl and fluoroC₁₋₃alkoxy groups, for example, CF₃, CH₂CH₂F, CH₂CHF₂, CH₂CF₃, OCF₃, OCH₂CH₂F, OCH₂CHF₂ or OCH₂CF₃, and most especially CF₃, OCF₃ and OCH₂CF₃.

The cycloalkyl groups referred to herein may represent, for example, cyclopropyl, cyclobutyl, cyclopentyl or cyclohexyl. A suitable cycloalkylalkyl group may be, for example, cyclopropylmethyl.

Similarly cycloalkoxy groups referred to herein may represent, for example, cyclopropoxy or cyclobutoxy.

As used herein, the terms “alkenyl” and “alkynyl” as a group or part of a group means that the group is straight or branched. Examples of suitable alkenyl groups include vinyl and allyl. A suitable alkynyl group is propargyl.

As used herein, the term “heteroaryl” as a group or part of a group means a heteroaromatic ring selected from pyrrolyl, furanyl, thienyl, pyridyl, pyrazolyl, imidazolyl, oxazolyl, isoxazolyl, thiazolyl, isothiazolyl, pyrazinyl, pyrimidinyl, pyridazinyl, triazolyl, oxadiazolyl, thiadiazolyl, triazinyl, tetrazolyl, indolyl, benzofuranyl, benzthiophenyl, benzimidazolyl, benzisoxazolyl, benzoxazolyl, benzthiazolyl or benzisothiazolyl. Particularly preferred examples of “heteroaryl” groups include pyridyl, indolyl and triazolyl, especially 2-pyridyl, 3-pyridyl, 2-indolyl and 1,2,4-triazol-3-yl.

When used herein the term “halogen” means fluorine, chlorine, bromine and iodine. The most apt halogens are fluorine and chlorine of which fluorine is preferred, unless otherwise stated.

A particularly preferred class of compounds of the present invention are the compounds of formula (Ia)

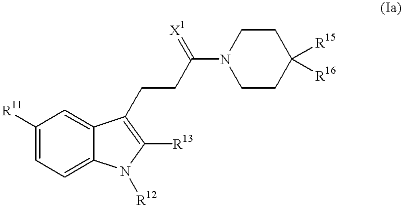

or a pharmaceutically acceptable salt thereof wherein

R¹¹ represents a chlorine or bromine atom or a methyl, vinyl, N-pyrrolidinyl, N-piperidinyl, N-morpholino, methoxycarbonyl, acetyl, 3-pyridyl or 2-furyl group;

R¹² represents a hydrogen atom or a methyl or acetyl group;

R¹³ represents 2-pyridyl, 3-pyridyl, unsubstituted phenyl, or phenyl substituted by a halogen atom;

R¹⁵ represents cyclohexyl, phenyl, 2-indolyl, CH₂phenyl, CH₂CH₂phenyl, CO(p-methoxyphenyl), C(OH)(phenyl)₂, NR^{c}R^{d} or CH₂NR^{c}R^{d} (where R^{c} and R^{d} each independently represent hydrogen, methyl, COCH₃, COCH₂CH₃, SO₂CH₃ or phenyl, or R^{c} and R^{d}, together with the nitrogen atom to which they are attached, form a piperidine ring) and wherein each phenyl group is optionally substituted by one or two substituents selected from fluorine, chlorine, bromine, methyl, methoxy, trifluoromethoxy or SO₂CH₃;

R¹⁶ represents hydrogen, fluorine, cyano, NR^{c}R^{d} (where R^{c} and R^{d} each independently represent hydrogen or methyl), NHCOCH₃, CH₂NHCOCH₃, CO₂H, CO₂CH₃, OH or CH₂OH; or

R¹⁵ and R¹⁶ together are so linked as to form a 5- or 6-membered ring optionally substituted by ═O, and optionally containing a double bond, which ring optionally contains in the ring an oxygen or sulfur atom or 1 or 2 NH groups, and to which ring is either fused or attached a benzene ring, which benzene ring is optionally substituted by methyl or SO₂CH₃; and

X¹ represents an oxygen atom or ═NH.

Particularly preferred compounds of formula (Ia) are those wherein R¹¹ represents chlorine or methyl.

Another preferred class of compounds of formula (Ia) is that wherein R¹² represents hydrogen or methyl.

A further preferred class of compounds of formula (Ia) is that wherein R¹³ represents 2-pyridyl, 3-pyridyl, phenyl, 4-fluorophenyl, 4-chlorophenyl or 4-bromophenyl, especially phenyl or 4-chlorophenyl.

A further preferred class of compounds of formula (Ia) is that wherein R¹⁵ represents cyclohexyl, phenyl, benzyl, 4-chlorophenyl, 3-trifluoromethylphenyl, NH(phenyl), N(CH₃)(phenyl) or N(COCH₂CH₃)(phenyl).

Another further preferred class of compound of formula (Ia) is that wherein R¹⁶ represents hydrogen, fluorine, hydroxy or CO₂CH₃.

In a further aspect of the present invention, the compounds of formula (I) may be prepared in the form of a pharmaceutically acceptable salt, especially an acid addition salt.

For use in medicine, the salts of the compounds of formula (I) will be non-toxic pharmaceutically acceptable salts. Other salts may, however, be useful in the preparation of the compounds according to the invention or of their non-toxic pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulphonic acid, maleic acid, succinic acid, acetic acid, citric acid, tartaric acid, carbonic acid, phosphoric acid or sulphuric acid. Salts of amine groups may also comprise quaternary ammonium salts in which the amino nitrogen atom carries a suitable organic group such as an alkyl, alkenyl, alkynyl or aralkyl moiety.

The salts may be formed by conventional means, such as by reacting the free base form of the product with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention includes within its scope solvates of the compounds of formula (I) and salts thereof, for example, hydrates.

The compounds according to the invention may have at least one asymmetric centre, and may exist both as enantiomers and as diastereoisomers. It is to be understood that all such isomers and mixtures thereof are encompassed within the scope of the present invention.

The present invention further provides pharmaceutical compositions comprising one or more compounds of formula (I) in association with a pharmaceutically acceptable carrier or excipient.

Preferably the compositions according to the invention are in unit dosage forms such as tablets, pills, capsules, powders, granules, solutions or suspensions, or suppositories, for oral, parenteral or rectal administration, or administration by inhalation or insufflation. Oral compositions such as tablets, pills, capsules or wafers are particularly preferred.

A more detailed description of pharmaceutical compositions that are suitable for the formulation of compounds of the present invention is disclosed in U.S. Pat. No. 6,071,927, the content of which is incorporated herein by reference (see in particular, column 8, line 50 to column 10, line 4).

The present invention further provides a process for the preparation of a pharmaceutical composition comprising a compound of formula (I), which process comprises bringing a compound of formula (I) into association with a pharmaceutically acceptable carrier or excipient.

The compounds of formula (I) are of value in the treatment of a wide variety of clinical conditions which are characterised by the presence of an excess of tachykinin, in particular substance P, activity. A comprehensive listing of clinical conditions, uses and methods of treatment for which the compounds of the present invention will be useful is disclosed in U.S. Pat. No. 6,071,927, the content of which is incorporated herein by reference (see, in particular, column 10, line 14 to column 22, line 18).

In particular, the compounds of the present invention are useful in the treatment of a variety of disorders of the central nervous system. Such disorders include mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; and anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalised anxiety disorders.

The compounds of the present invention are also particularly useful in the treatment of nociception and pain. Diseases and conditions in which pain predominates, include soft tissue and peripheral damage, such as acute trauma, osteoarthritis, rheumatoid arthritis, musculo-skeletal pain, particularly after trauma, spinal pain, myofascial pain syndromes, headache, migraine, episiotomy pain, and burns.

The compounds of the present invention are also particularly useful in the treatment of respiratory diseases, particularly those associated with excess mucus secretion, such as chronic obstructive airways disease, bronchopneumonia, chronic bronchitis, cystic fibrosis and asthma, adult respiratory distress syndrome, and bronchospasm; in the treatment of inflammatory diseases such as inflammatory bowel disease, psoriasis, fibrositis, osteoarthritis, rheumatoid arthritis, pruritis and sunburn; and in the treatment of allergic disorders such as eczema and rhinitis.

The compounds of the present invention are also particularly useful in the treatment of gastrointestinal (GI) disorders, including inflammatory disorders and diseases of the GI tract such as ulcerative colitis, Crohn's disease and irritable bowel syndrome.

The compounds of the present invention are also particularly useful in the treatment of emesis, including acute, delayed or anticipatory emesis, such as emesis induced by chemotherapy, radiation, toxins, pregnancy, vestibular disorders, motion, surgery, migraine, and variations in intercranial pressure. Most especially, the compounds of formula (I) are of use in the treatment of emesis induced by antineoplastic (cytotoxic) agents, including those routinely used in cancer chemotherapy; by radiation including radiation therapy such as in the treatment of cancer; and in the treatment of post-operative nausea and vomiting.

The excellent pharmacological profile of the compounds of the present invention offers the opportunity for their use in therapy at low doses thereby minimising the risk of unwanted side effects.

In the treatment of the conditions associated with an excess of tachykinins, a suitable dosage level is about 0.001 to 50 mg/kg per day, in particular about 0.01 to about 25 mg/kg, such as from about 0.05 to about 10 mg/kg per day.

For example, in the treatment of conditions involving the neurotransinission of pain sensations, a suitable dosage level is about 0.001 to 25 mg/kg per day, preferably about 0.005 to 10 mg/kg per day, and especially about 0.005 to 5 mg/kg per day. The compounds may be administered on a regimen of 1 to 4 times per day, preferably once or twice per day.

In the treatment of emesis, a suitable dosage level is about 0.001 to 10 mg/kg per day, preferably about 0.005 to 5 mg/kg per day, and especially 0.01 to 3 mg/kg per day. The compounds may be administered on a regimen of 1 to 4 times per day, preferably once or twice per day.

In the treatment of psychiatric disorders, a suitable dosage level is about 0.001 to 10 mg/kg per day, preferably about 0.005 to 5 mg/kg per day, and especially 0.01 to 3 mg/kg per day. The compounds may be administered on a regimen of 1 to 4 times per day, preferably once or twice per day.

It will be appreciated that the amount of a compound of formula (I) required for use in any treatment will vary not only with the particular compounds or composition selected but also with the route of administration, the nature of the condition being treated, and the age and condition of the patient, and will ultimately be at the discretion of the attendant physician.

As used herein, the term “treatment” includes prophylactic use to prevent the occurrence or recurrence of any of the aforementioned conditions.

Several methods for preparing the compounds of the present invention are illustrated in the following schemes and Examples wherein R¹, R², R³, R⁴, R⁵, R⁶, X and Y are as defined above unless otherwise specified.

### Abbreviations used in the Schemes

Reagents

| | |
|---|---|
| Ac₂O | acetyl acetate |
| AcCl | acetyl chloride |
| AcOH | acetic acid |
| (RS)-BINAP | (R,S)-2,2′-bis(diphenylphosphino)-1,1′-binaphthyl |
| BH₃.THF | borane-tetrahydrofuran complex |
| BrCH₂CN | bromoacetonitrile |
| C₆F₅CH₂OH | pentafluorobenzyl alcohol |
| CBr₄ | carbontetrabromide |
| CDI | carbonyldiimidazole |
| DIAD | diisopropyl azodicarboxylate |
| DIBAL-H | diisobutylaluminium hydride |
| DIC | 2-dimethylaminoisopropyl chloride hydrochloride |
| DMAP | 4-dimethylaminopyridine |
| DMSO | dimethylsulfoxide |
| (DPPB)PdCl₂ | [1,4-butanediylbis(diphenylphosphine)] |
| | dichloropalladium |
| EDC | 1-(3-dimethylaminopropyl)-3-ethylcarbodiimide |
| | hydrochloride |
| Et₃N | triethylamine |
| EtOH | ethanol |
| H₂, Pd-C | catalytic hydrogenation using palladium on carbon |
| HCl | hydrochloric acid |
| HOBT | 1-hydroxybenzotriazole hydrate |
| iPr₂EtN | diisopropylethylamine |
| K₂CO₃ | potassium carbonate |
| KCN | potassium cyanide |
| KHMDS | potassium bis(trimethylsilyl)amide |
| KOH | potassium hydroxide |
| KOt-Bu | potassium tert-butoxide |
| LiOH | lithium hydroxide |
| MeI | methyl iodide |
| MeOH | methanol |
| Na(AcO)₃BH | sodium triacetoxyborohydride |
| NaH | sodium hydride |
| NaOH | sodium hydroxide |
| n-BuLi | n-butyl lithium |
| NCCO₂Me | methyl cyanoformate |
| Pd(Ph₃P)₄ | tetrakis(triphenylphosphine)palladium (0) |
| Pd₂(DBA)₃ | tris(dibenzylideneacetone)dipalladium (0) |
| Pd-C | palladium on carbon |
| Ph₃P | triphenylphosphine |
| TFA | trifluoroacetic acid |
| TMSBr | bromotrimethylsilane |
| ZnCl₂ | zinc chloride |

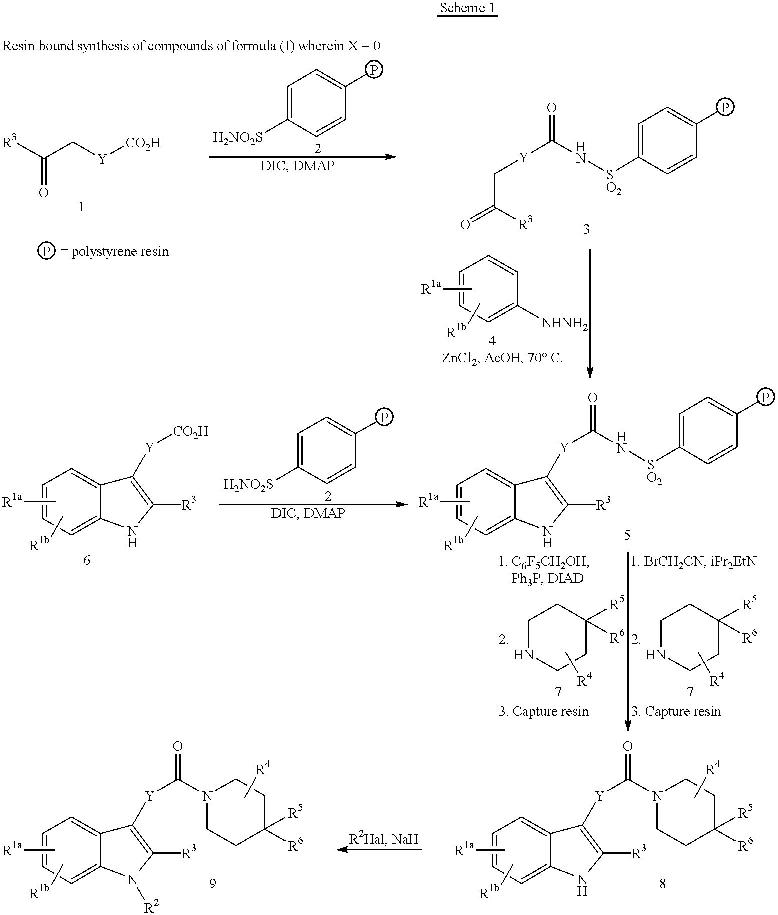

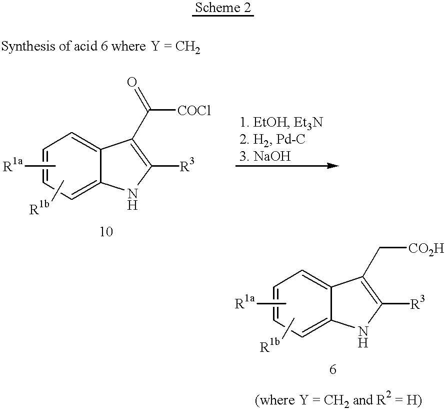

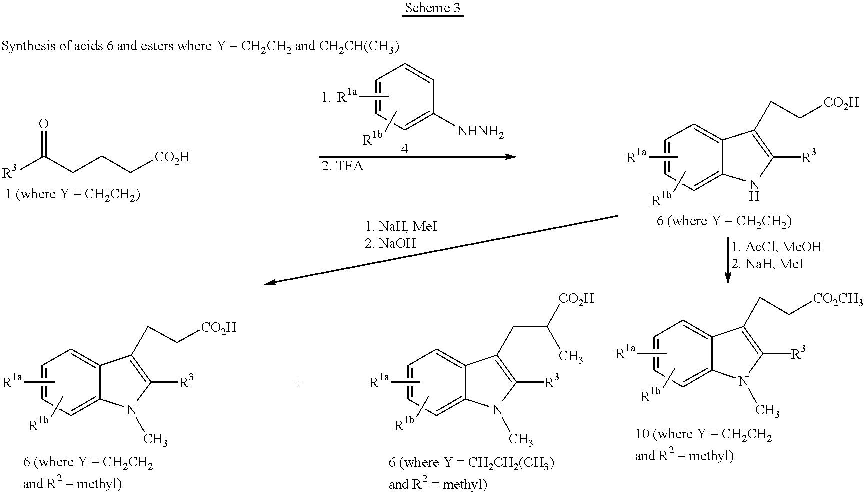

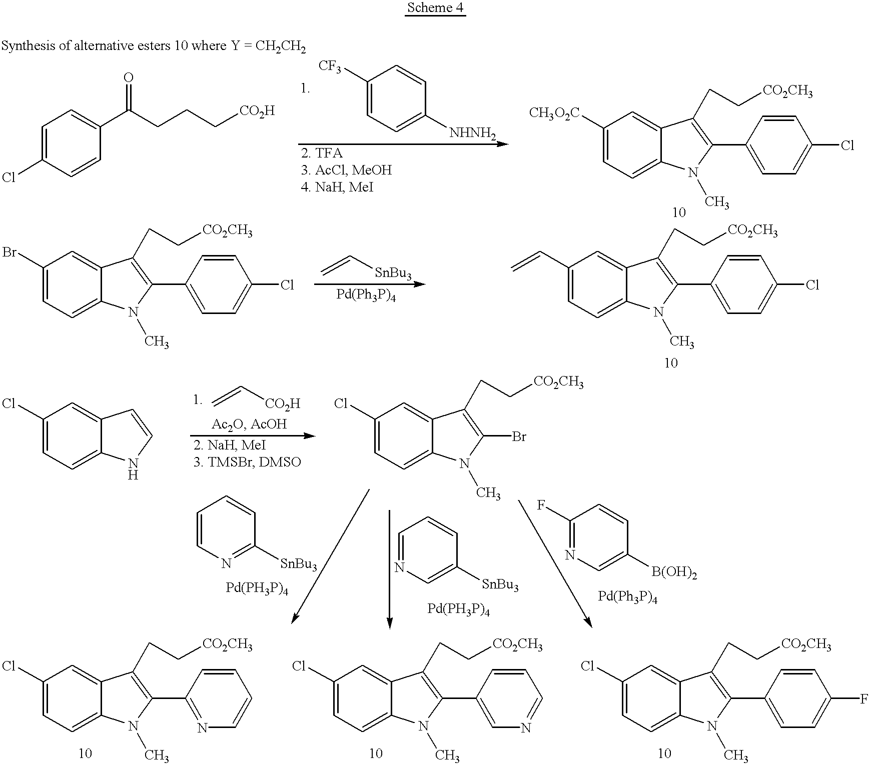

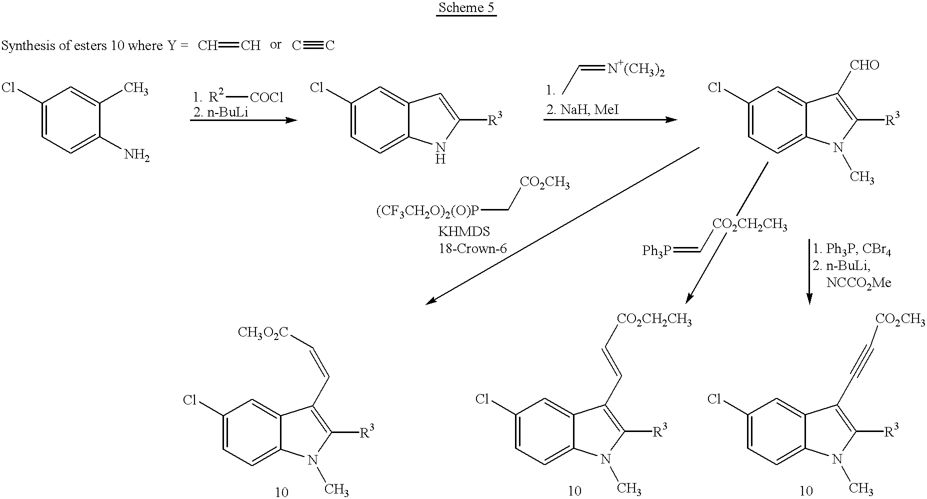

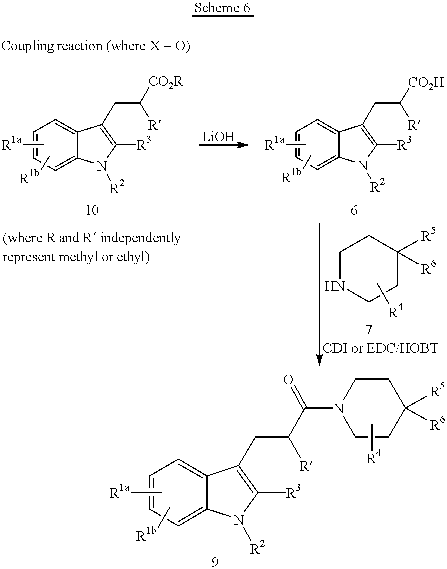

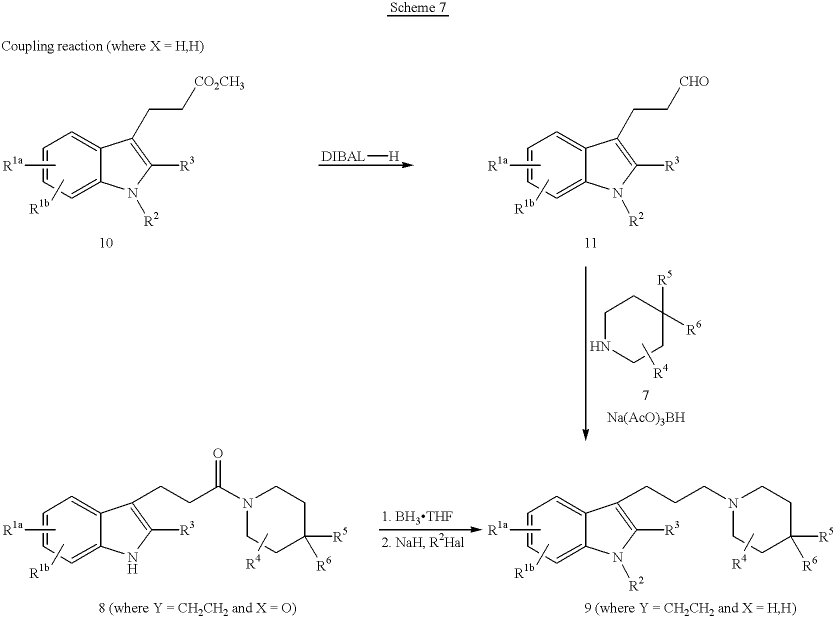

The compounds of the present invention in which X═O may be prepared by the general route outlined in Scheme 1 or by methods analogous thereto. Thus, the appropriate carboxylic acid 1 is loaded onto a resin 2, for example, 4-sulfamylbutyryl AM resin (Novabiochem), using a suitable coupling agent such as 1,3-diisopropylcarbodiimide. The reaction is conveniently effected in the presence of an organic base such as 4-dimethylaminopyridine. The resultant loaded resin 3 may then be reacted with phenyl hydrazine or with a variety of mono- or disubstituted phenyl hydrazines 4 to prepare the indole intermediate 5 which, at this stage, is still bound to the resin. Reaction with the hydrazine is an example of the well-known Fischer indole synthesis, conveniently effected in glacial acetic acid in the presence of a suitable catalyst, for example, a Lewis acid such as zinc chloride.

An alternative route to the resin bound indoles 5 involves the coupling of a preformed indole carboxylic acid 6 with the resin 2 using the conditions described above.

Preparation of the compounds of formula (I) is completed by an exchange reaction that liberates the resin and introduces the substituted piperidine moiety. This exchange reaction may be effected using a variety of conditions such as pentafluorobenzyl alcohol, triphenylphosphine and diisopropyl azodicarboxylate to introduce the substituted piperidine 7. Alternatively, the reaction may be effected in the presence of diisopropylethylamine and bromoacetonitrile.

The compound of formula (I) 8 is readily modified on the indole nitrogen using conventional methodology. Thus, for example, where R² is an alkyl group, reaction with an appropriate alkyl halide in the presence of a hydride, affords further compounds of formula (I) 9.

The indole-3-acetic acid intermediates 6 (where Y is CH₂) may be prepared by the general route outlined in Scheme 2 or by methods analogous thereto. Thus, an appropriately substituted α-oxo-indole-3-acetyl chloride 10 may be converted to the corresponding ester by reaction with, for example, ethanol, in the presence of a base, such as triethylamine. The resultant oc-oxo ester is then reduced using, for example, catalytic hydrogenation using a transition metal catalyst such as palladium on carbon, followed by hydrolysis using, for example, a hydroxide such as sodium hydroxide, to afford the acetic acid compound 6.

The indole-3-propanoic acid intermediates 6 (where Y is CH₂CH₂) may be prepared by the general route outlined in Scheme 3 or by methods analogous thereto. Thus, an appropriately substituted δ-oxopentanoic acid 1 (where Y is CH₂CH₂) may be reacted with phenyl hydrazine or with a mono- or disubstituted phenyl hydrazine under conventional conditions for the Fischer indole synthesis or by heating the mixture at reflux in the presence of trifluoroacetic acid. The reaction is conveniently effected in a solvent, for example, triethylamine.

The indole-3-propionic acid 6 in Scheme 3 is readily modified on the indole nitrogen using conventional methodology described above. Similarly, the linking chain Y may be substituted using conventional methylation conditions. The acid function may also be converted into its corresponding ester (e.g. the methyl ester) 10 (where Y is CH₂CH₂ and R² is methyl).

Other indole-3-carboxylic acids in which Y is (CH₂)₃ or (CH₂)₄ may be prepared by methods analogous to those described in Scheme 3 or by other methods well known to a person of ordinary skill in the art.

In an alternative method, indole-3-propionate ester intermediates 10 (where Y is CH₂CH₂) may be prepared by the general routes outlined in Scheme 4 or by methods analogous thereto.

Further ester intermediates 10 (where Y is CH═CH or C≡C) may be prepared by the general routes outlined in Scheme 5 or by methods analagous thereto. In particular, a common 3-formyl indole intermediate 11 may be reacted in a variety of ways to introduce a cis-CH═CHCO₂Me, trans-CH═CHCO₂Et or C≡CCO₂Me substituent.

In an alternative method, compounds of the present invention in which X═O may be prepared by the general route outlined in Scheme 6 or by methods analogous thereto. A 2-aryl-indole-3-carboxylate ester intermediate 10 may be dealkylated to the corresponding carboxylic acid 6 using, for example, lithium hydroxide. The acid 6 may be coupled to a piperidine intermediate 7 using conventional coupling conditions such as treating the carboxylic acid with 1,1-carbonyldiimidazole, followed by reaction with the piperidine. The reaction is conveniently effected in a suitable solvent such as an ether, for example, tetrahydrofuran. The treatment with CDI is preferably effected at the reflux temperature of the solvent whereas reaction with the piperidine is preferably effected at about room temperature.

Alternative coupling conditions comprise mixing a 2-aryl-indole-3-carboxylic acid 6 and the piperidine 7 with 1-hydroxybenzotriazole, followed by addition of 1-(3-dimethylaminopropyl)-3-ethylcarbodiimide hydrochloride. The reaction is conveniently effected in a suitable solvent such as an ether, for example, tetrahydrofuran, and preferably at about room temperature, to afford the compound of formula (I) 9.

In a further alternative method, compounds of the present invention in which X is two hydrogen atoms may be prepared by the general route outlined in Scheme 7 or by methods analogous thereto. A 2-aryl-indole-3-carboxylate ester intermediate 10 may be reduced to the corresponding aldehyde 11 using a suitable reducing agent such as diisobutylaluminium hydride. The reaction is conveniently effected in a suitable solvent such as a halogenated hydrocarbon, for example, dichloromethane, or a hydrocarbon, for example, hexane, or a mixture thereof. The aldehyde 11 may then be coupled to the piperidine 7 using conventional coupling conditions such as mixing with sodium triacetoxyborohydride. The reaction is conveniently effected in glacial acetic acid and a halogenated hydrocarbon, for example, 1,2-dichloroethane.

In an alternative method, compounds of the present invention in which X is two hydrogen atoms may be prepared from a corresponding compound of formula (I) where X is an oxygen atom 8 according to the general route outlined in Scheme 7, or by methods analogous thereto. Reduction of the ketone may be effected using conventional conditions such as mixing with a borane tetrahydrofuran complex, conveniently in tetrahydrofuran as the solvent.

The piperidine intermediates 7 may be prepared by conventional procedures using the methods described in the specific Examples herein or using methods analogous thereto.

It will be appreciated that, where appropriate, a combination of the general methodology described in Schemes 1 to 7 may be applied to prepare further compounds of the present invention.

The compounds of formula (I) prepared according to the methods described above may be isolated and purified in a conventional manner, for example, extraction, precipitation, fractional crystallization, recrystallization, chromatography or a combination thereof.

Although the reaction schemes described herein are reasonably general, it will be understood by those skilled in the art of organic synthesis that one or more functional groups present in a given compound of formula (I) may render the molecule incompatible with a particular synthetic sequence.

In such a case an alternative route, an altered order of steps, or a strategy of protection and deprotection may be employed. In all cases the particular reaction conditions, including reagents, solvent, temperature, and time, should be chosen so that they are consistent with the nature of the functionality present in the molecule.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene and P. G. M. Wuts, Protective Groups in Organic Synthesis, John Wiley & Sons, 1991. The protecting groups may be removed at a convenient subsequent stage using methods known from the art.

The exemplified compounds of this invention were tested by the methods set out at pages 36 to 39 of International Patent Specification No. WO 93/01165. The compounds were found to be active with IC₅₀ at the NK₁ receptor of less than 100 nM on said test method.

The following non-limiting Examples serve to illustrate the preparation of compounds of the present invention:

### DESCRIPTION 1

### Loaded Resin

1,3-Diisopropylcarbodiimide (3.47 g) was added to 4-bromo-6-oxobenzenepentanoic acid (J. Org. Chem. 1948, 13, 284; J. Org. Chem. 1984, 49, 3170; 14.91 g) in dichloromethane (55 mL) and the mixture was stirred at room temperature for 30 min. The mixture was added to 4-sulfamylbutyryl AM resin (Novabiochem, product no. 01-64-0152, 1 mmol/g loading, 5.5 g), 4-dimethylaminopyridine (671 mg) was added and the mixture was stirred at room temperature for 18 h. The mixture was filtered and the resin was washed with dimethylformamide (50 mL), dichloromethane (50 mL), methanol (50 mL) and ether (50 mL) and dried in vacuo. A dispersion of (4-methylphenyl)hydrazine hydrochloride (9.2 g) in glacial acetic acid (135 mL) then zinc chloride (10.8 g) were added and the mixture was heated to 75 ° C. for 18 h. The mixture was cooled to room temperature, filtered and washed with glacial acetic acid-tetrahydrofuran (1:1, 50 mL), dichloromethane (50 mL), dimethylformamide (50 mL), methanol (50 mL) and ether (50 mL) and dried in vacuo.

### DESCRIPTION 2

### Determination of Resin Loading by Preparation of N,N-Dimethyl- [2-(4-Bromophenyl)-5-Methyl-1H-Indol-3-yl]Propanamide

Diisopropylethylamine (65 mg) and bromoacetonitrile (288 mg) were added to the resin of Description 1 (100 mg) in N-methylpyrrolidinone (1 mL) and the mixture was allowed to stand at room temperature for 18 h. The mixture was filtered and the resin was washed with N-methylpyrrolidinone (5 mL) and tetrahydrofuran (5 mL). A solution of dimethylamine in tetrahydrofuran (2M, 2 mL) was added and the mixture was allowed to stand at room temperature for 18 h. The mixture was filtered and the filtrate was collected. The solvent was evaporated under reduced pressure and the residue was dried in vacuo to give the title compound as a pale brown solid (20 mg), consistent with a resin loading of 0.52 mmol/g prior to activation and cleavage.

### DESCRIPTION 3

### Loaded Resin

1,3-Diisopropylcarbodiimide (1.21 g) was added to 5-chloro-2-(4-chlorophenyl)-1H-indole-3-propanoic acid (Description 6, 6.4 g, 19.2 mmol) in tetrahydrofuran-dichloromethane (1:1, 80 mL) and the mixture was stirred at room temperature for 30 min. The mixture was added to 4-sulfamylbutyryl AM resin (Novabiochem, product no. 01-64-0152, 1 mmol/g loading, 8.3 g), 4-dimethylaminopyridine (292 mg) was added and the mixture was stirred at room temperature for 16 h. The mixture was filtered and the resin was washed with tetrahydrofuran-dichloromethane (1:1), methanol (50 mL) and tetrahydrofuran (3×) and dried in vacuo.

### DESCRIPTION 4

### Determination of Resin Loading by Preparation of N,N-Dimethyl-[5-Chloro-2-(4-Chlorophenyl)-1H-Indol-3-yl]Propanamide

The loading of the resin of Description 3 was determined to be 0.51 mmol/g by the method of Description 2.

### DESCRIPTION 5

### 5-Methyl-2-Phenyl-1H-Indole-3-Propanoic Acid

Triethylamine (1.4 mL, 10 mmol) was added to a stirred suspension of δ-oxobenzenepentanoic acid (1.92 g, 10 mmol) and (4-methylphenyl)hydrazine hydrochloride (1.59 g, 10 mmol) in ethanol (16 mL) and the mixture was stirred at room temperature for 4 h. Ether (100 mL) was added, the mixture was filtered and the solvent was evaporated under reduced pressure. The residue was added slowly to trifluoroacetic acid (15 mL) and the mixture was heated under reflux for 2 h. The mixture was cooled, water (100 mL) was added and the mixture was extracted with ethyl acetate (100 mL). The organic fraction was washed with brine (30 mL), dried (MgSO₄) and the volume was reduced to Ca. 10 mL by evaporation under reduced pressure. The precipitate was collected and recrystallized from ether to give the title compound as a pale solid (1.51 g, 54%). ¹H NMR (360 MHz, DMSO-d₆) δ2.39 (3H, s), 2.50-2.58 (2H, m), 3.03-3.09 (2H, m), 6.91-6.95 (1H, m), 7.23-7.25 (1H, m), 7.34-7.40 (2H, m), 7.47-7.63 (4H, m), 11.4 (1H, br s), and 12.25 (1H, br s). n/z (ES⁺) 280 (M+1).

### DESCRIPTION 6

### 5-Chloro-2-(4-Chlorophenyl)-1H-Indole-3-Propanoic Acid

Prepared from 4-chloro-δ-oxobenzenepentanoic acid and (4-chlorophenyl)hydrazine hydrochloride according to the method of Description 5. ¹H NMR (250 MHz, DMSO-d₆) δ2.55-2.61 (2H, m), 3.07-3.13 (2H, m), 7.16 (2H, d, J 12.6 Hz), 7.41 (2H, d, J 12.6 Hz), 7.62-7.71 (3H, m), 11.52 (1H, br s), and 12.19 (1H, br s). m/z (ES⁺) 331, 333 (M+1).

### DESCRIPTION 7

### 5-Bromo-2-(4-Chlorophenyl)-1H-Indole-3-Propanoic Acid

Prepared from 4-chloro-δ-oxobenzenepentanoic acid and (4-bromophenyl)hydrazine hydrochloride according to the method of Description 5. ¹H NMR (360 MHz, DMSO-d₆) δ12.11 (1H, br s), 11.47 (1H, s), 7.78 (1H, d, J 1.8 Hz), 7.66-7.57 (4H, m), 7.32 (1H, d, J 8.5 Hz), 7.22 (1H, dd, J 1.8, 8.5 Hz), 3.06 (2H, t, J 8.0 Hz), and 2.53 (2H, t, J 8.0 Hz).

### DESCRIPTION 8

### Methyl 2-(4-Chlorophenyl)-5-(Trifluoromethyl)-1H-Indole-3-Propanoate

A mixture of 4-chloro-δ-oxobenzenepentanoic acid (2.0 g, 8.8 mmol) and 4-(trifluoromethyl)phenylhydrazine (5.0 g, 28.4 mmol) in trifluoroacetic acid (100 mL) was stirred at 65° C. for 3 days. The mixture was cooled and the solvent was evaporated under reduced pressure. Water (50 mL) and ethyl acetate (50 mL) were added, the layers were separated and the aqueous layer was extracted with ethyl acetate (50 mL). The combined organic fractions were washed with brine (50 mL), dried (MgSO₄) and the solvent was evaporated under reduced pressure to give an oil (4.75 g). A portion (1.75 g) was dissolved in methanol (10 mL), acetyl chloride (102 μL, 1.43 mmol) was added and the mixture was heated under reflux for 3 h. The mixture was cooled and the solvent was evaporated under reduced pressure. Water (25 mL) and ethyl acetate (25 mL) were added, the layers were separated and the aqueous layer was extracted with ethyl acetate (3×50 mL). The combined organic fractions were washed with aqueous sodium carbonate (10%, 6×25 mL) and brine (25 mL), dried (MgSO₄) and the solvent was evaporated under reduced pressure. The residue was purified by flash column chromatography on silica gel, eluting with hexane-EtOAc (4:1 increasing to 1:2) to give the crude title compound as a yellow solid (50 mg, 4%). ¹H NMR (360 MHz, DMSO-d₆) (Contains approximately 30% impurities) δ11.64 (1H, s), 8.23 (1H, d, J 1.5 Hz), 7.75 (1H, dd, J 8.4, 1.5 Hz), 7.67 (2H, d, J 8.6 Hz), 7.60 (2H, d, J 8.6 Hz), 7.41 (1H, d, J 8.4 Hz), 3.53 (3H, s), 3.16 (2H, t, J 7.8 Hz), and 2.64 (2H, t, J 7.8 Hz).

### DESCRIPTION 9

### Methyl 5-Bromo-2-(4-Chlorophenyl)-1H-Indole-3-Propanoate

Acetyl chloride (130 μL, 1.82 mmol) was added to a solution of 5-bromo-2-(4-chlorophenyl)-1H-indole-3-propanoic acid (Description 7, 460 mg, 1.21 mmol) in methanol (15 mL) and the mixture was heated under reflux for 8 h. The mixture was cooled and the solvent was evaporated under reduced pressure. Water was added and the mixture was extracted with ethyl acetate. The organic fraction was washed with brine, dried (MgSO₄) and the solvent was evaporated under reduced pressure to give the title compound as an orange solid (400 mg, 84%). ¹H NMR (250 MHz, DMSO-d₆) δ11.50 (1H, s), 7.77 (1H, m), 7.66-7.57 (4H, m), 7.32 (1H, d, J 8.5 Hz), 7.22 (1H, m), 3.52 (3H, s), 3.09 (2H, t, J 7.7 Hz), and 2.62 (2H, t, J 7.7 Hz).

### DESCRIPTION 10

### Methyl 5-Chloro-2-(4-Chlorophenyl)-1-Methyl-1H-Indole-3-Propanoate

Sodium hydride (60% suspension in mineral oil, 4.5 g, 113 mmol) was added in portions to a stirred, cooled (0° C.) solution of 5-chloro-2-(4-chlorophenyl)-1H-indole-3-propanoic acid (Description 6, 7.5 g, 22.5 mmol) in dimethylformamide (50 mL) and the mixture was stirred at room temperature for 20 min. The mixture was cooled to 0° C., iodomethane (7 mL, 113 mmol) was added dropwise and the mixture was stirred at room temperature for 45 min. Ice cold water (500 mL) was added and the mixture was extracted with ether (3×400 mL). The combined organic fractions were washed with water (3×250 mL) and brine (250 mL), dried (MgSO₄) and the solvent was evaporated under reduced pressure to give an orange solid (8.6 g). A sample (1.0 g) was dissolved in hot methanol and decanted from insoluble material. The solvent was evaporated under reduced pressure and the residue was triturated with hexane. The solid was collected and dried in vacuo to give the title compound (0.43 g, 45%). ¹H NMR (360 MHz, CDCl₃) 6 7.57 (1H, d, J 1.7 Hz), 7.48 (2H, d, J 8.4 Hz), 7.30 (2H, d, J 8.4 Hz), 7.24 (1H, d, J 8.7 Hz), 7.20 (1H, dd, J 8.7, 1.7 Hz), 3.61 (3H, s), 3.53 (3H, s), 2.97 (2H, t, J 8.0 Hz), and 2.53 (2H, t, J 8.0 Hz).

### DESCRIPTION 11

### 5-Chloro-2-(4-Chlorophenyl)-1-Methyl-1H-Indole-3-Propanoic Acid and (RS)-α-Methyl-[5-Chloro-2-(4-Chlorophenyl)-1-Methyl-1H-Indole]-3-Propanoic Acid

Sodium hydride (60% suspension in mineral oil, 5.98 g) was added in portions to a stirred, cooled (0° C.) solution of 5-chloro-2-(4-chlorophenyl)-1H-indole-3-propanoic acid (Description 6, 10 g, 30 mmol) in dimethylformamide (100 mL) and the mixture was stirred at 0° C. for 30 min. Iodomethane (9 mL) was added in one portion and the mixture was stirred at room temperature for 30 min. Water (1.5 L) was added and the mixture was extracted with ether (3×400 mL). The combined organic fractions were dried (MgSO₄) and the solvent was evaporated under reduced pressure. The residue was dissolved in methanol (240 mL) and aqueous sodium hydroxide (4M, 60 mL) was added. The mixture was heated under reflux for 1 h., cooled and the pH was adjusted to 1.0 with hydrochloric acid (2M). The layers were separated and the aqueous layer was extracted with ethyl acetate. The combined organic fractions were dried (MgSO₄) and the solvent was evaporated under reduced pressure. The residue was triturated with hexane and the solid was collected and dried in vacuo to give 5-chloro-2-(4-chlorophenyl)-1-methyl-1H-indole-3-propanoic acid (7.5 g, 72%). ¹H NMR (360 MHz, CDCl₃) δ2.54-2.59 (2H, m), 2.96-3.00 (2H, m), 3.53 (3H, s), 7.18-7.30 (4H, m), 7.44-7.48 (2H, m), and 7.58 (1H, d, J 1.8 Hz). The mother liquors were evaporated under reduced pressure and the residue was purified by flash column chromatography on silica gel, eluting with hexane/EtOAc (80:20 increasing to 50:50) to give (RS)-α-methyl-[5-chloro-2-(4-chlorophenyl)-1-methyl-1H-indole]-3-propanoic acid (1.27 g, 12%). ¹H NMR (360 MHz, CDCl₃) δ1.01 (3H, d, J 6.4 Hz), 2.74 (2H, m), 3.12 (1H, m), 3.52 (3H, s), 7.19 (1H, dd, J 8.6, 1.8 Hz), 7.24 (1H, d, J 8.6 Hz), 7.28 (2H, d, J 8.4 Hz), 7.45 (2H, d, J 8.4 Hz) and 7.58 (1H, d, J 1.8 Hz).

### DESCRIPTION 12

### Methyl 2-(4-Chlorophenyl)-1-Methyl-5-(Methoxycarbonyl)-1H-Indole-3-Propanoate

Sodium hydride (60% dispersion in mineral oil, 8 mg, 0.2 mmol) was added to a solution of methyl 2-(4-chlorophenyl)-5-(trifluoromethyl)-1H-indole-3-propanoate (Description 8, 50 mg, 0.13 mmol) in dimethylformamide (7 mL) and the mixture was stirred at room temperature for 10 min. Iodomethane (12 μl, 0.2 mmol) was added and the mixture was stirred at room temperature for 5 min. Water (10 mL) and ethyl acetate (10 mL) were added, the layers were separated and the aqueous layer was extracted with ethyl acetate (15 mL). The combined organic fractions were washed with water (25 mL) and brine (25 mL), dried (MgSO₄) and the solvent was evaporated under reduced pressure to give the title compound (35 mg, 69%).¹H NMR (360 MHz, DMSO-d₆) δ8.27 (1H, d, J 1.0 Hz), 7.83 (1H, dd, J 9.4, 1.0 Hz), 7.63 (2H, d, J 8.4 Hz), 7.58 (1H, d, J 9.4 Hz), 7.52 (2H, d, J 8.4 Hz), 3.87 (3H, s), 3.61 (3H, s), 3.59 (3H, s), 2.95 (2H, t, J 7.8 Hz), and 2.53 (2H, t, J 7.8 Hz).

### DESCRIPTION 13

### Methyl 5-Bromo-2-(4-Chlorophenyl)-1-Methyl-1H-Indole-3-Propanoate

Prepared from methyl 5-bromo-2-(4-chlorophenyl)-1H-indole-3-propanoate (Description 9) according to the method of Description 12. ¹H NMR (360 MHz, DMSO-d₆) δ7.79 (1H, d, J 1.9 Hz), 7.63-7.60 (2H, m), 7.50-7.45 (3H, m), 7.30 (1H, dd, J 8.7, 1.9 Hz), 3.54 (3H, s), 3.50 (3H, s), 2.88 (2H, t, J 7.8 Hz), and 2.51 (2H, t, J 7.8 Hz).

### DESCRIPTION 14

### Methyl 2-(4-Chlorophenyl)-5-Ethenyl-1-Methyl-1H-Indole-3-Propanoate

Vinyltributyltin (281 mg, 0.89 mmol) and lithium chloride (188 mg, 4.43 mmol) were added to a solution of methyl 5-bromo-2-(4-chlorophenyl)-1-methyl-1H-indole-3-propanoate (Description 13, 300 mg, 0.74 mmol) in toluene (10 mL) and the mixture was degassed. Tetrakis(triphenylphosphine)palladium (0) (30 mg) was added and the mixture was degassed, then heated under reflux for 2.5 h. Further tetrakis(triphenylphosphine)palladium (0) (30 mg) was added and the mixture was degassed, then heated under reflux overnight. Further tetrakis(triphenylphosphine)palladium (0) (30 mg) was added and the mixture was degassed, then heated under reflux for 8 h. The mixture was filtered through a glass fibre pad and the solvent was evaporated under reduced pressure. The residue was dissolved in ethyl acetate (10 mL) and aqueous potassium fluoride (5%, 10 mL) was added. The mixture was stirred at room temperature for 1 h., filtered and the layers were separated. The organic fraction was washed with water and brine, dried (MgSO₄) and the solvent was evaporated under reduced pressure. The residue was purified by flash column chromatography on silica gel, eluting with hexane/EtOAc (10:1), to give the title compound (108 mg, 41%). ¹H NMR (360MHz, CDCl₃) δ7.60 (1H, m), 7.49-7.26 (6H, m), 6.87 (1H, dd, J 17.6, 10.9 Hz), 5.73 (1H, d, J 17.6 Hz), 5.12 (1H, d, J 10.9 Hz), 3.61 (3H, s), 3.54 (3H, s), 3.04-3.00 (2H, m), and 2.59-2.52 (2H, m).

### DESCRIPTION 15

### 4-Chloro-N-(4-Chloro-2-Methylphenyl)Benzamide

4-Chlorobenzoyl chloride (14.1 mL) was added dropwise over 10 min. to a stirred, cooled (0° C.) mixture of 4-chloro-2-methylbenzenamine (15 g) and triethylamine (22.2 mL) in dichloromethane (300 mL) and the mixture was stirred at room temperature for 2 h. Water (100 mL) was added and the dichloromethane was evaporated under reduced pressure. Ethyl acetate (1 L), aqueous sodium hydrogen carbonate (saturated, 200 mL) and water (200 mL) were added and the layers were separated. The solid was collected and the organic fraction was evaporated under reduced pressure. The residue was triturated with ether and the solid was collected. The solids were combined, triturated with ether and dried in vacuo to give the title compound (27.3 g). ¹H NMR (CDCl₃) δ7.87-7.77 (3H, m), 7.56 (1H, s), 7.50-7.45 (2H, d, J 8 Hz), 7.26-7.20 (2H, m), and 2.30 (3H, s).

### DESCRIPTION 16

### 5-Chloro-2-(4-Chlorophenyl)-1H-Indole

n-Butyllithium (1.6M in hexanes, 127 mL) was added dropwise over 30 min. to a stirred, cooled (−10 ° C.) solution of 4-chloro-N-(4-chloro-2-methylphenyl)benzamide (Description 15, 27.1 g) in THF (600 mL). The mixture was allowed to warm gradually to room temperature and stirred for 16 h. A further portion of n-butyllithium (1.6M in hexanes, 30 mL) was added and the mixture was stirred at room temperature for 2 h. A third portion of n-butyllithium (1.6M in hexanes, 30 mL) was added and the mixture was stirred at room temperature for 2 h. Hydrochloric acid (2.5M, 200 mL) was added and the mixture was stirred at room temperature for 18 h. The mixture was extracted with ethyl acetate (2×500 mL) and the combined organic fractions were dried (MgSO₄) and the solvent was evaporated under reduced pressure. The residue was purified by flash column chromatography on silica gel, eluting with hexane/EtOAc (90:10) to give the title compound as a yellow solid. ¹H NMR (CDCl₃) δ8.31 (1H, br s), 7.59-7.54 (3H, m), 7.46-7.40 (2H, m), 7.30 (1H, d, J 8.5 Hz), 7.15 (1H, dd, J 8.5, 2.0 Hz), and 6.74 (1H, br s).

### DESCRIPTION 17

### 5-Chloro-2-(4-Chlorophenyl)- 1H-Indole-3-Carboxaldehyde

A mixture of 5-chloro-2-(4-chlorophenyl)-1H-indole (Description 16, 3.10 g, 11.8 mmol) and N-(chloromethylene)-N-methylmethaniminium chloride (2.2 g) in dimethylformamide (100 mL) was heated at 75 ° C. for 16 h., cooled, poured into aqueous sodium hydroxide (2M, 500 mL) and ice (400 g) was added. After the ice had melted, the solid was collected, washed with water (20 mL) and dried in vacuo to give the title compound as an orange solid (3.05 g, 88%). ¹H NMR (DMSO-d₆) δ9.93 (1H, br s), 8.17 (1H, br s), 7.83 (2H, d, J 8 Hz), 7.66 (2H, d, J 8 Hz), 7.52 (1H, d, J 8.5 Hz), and 7.29 (1H, d, J 8.5 Hz).

### DESCRIPTION 18

### 5-Chloro-2-(4-Chlorophenyl)-1-Methyl-1H-Indole-3-Carboxaldehyde

Sodium hydride (60% dispersion in mineral oil, 310 mg) was added to a solution of 5-chloro-2-(4-chlorophenyl)-1H-indole-3-carboxaldehyde (Description 17, 1.07 g, 3.7 mmol) in dimethylformamide (10 mL). Iodomethane (305 μL) was added and the mixture was stirred at room temperature for 1 h. The mixture was poured into ice-water (100 mL) and the solid was collected and washed with water (20 mL). Toluene (40 mL) was added and evaporated under reduced pressure to give the title compound as a pale yellow solid (1.10 g, 98%). ¹H NMR (CDCl₃) δ9.70 (1H, s), 8.42 (1H, br s), 7.56 (2H, d, J 8.4 Hz), 7.43 (2H, d, J 8.4 Hz), 7.38-7.28 (2H, m), and 3.66 (3H, s).

### DESCRIPTION 19

### (E)-Ethyl [5-Chloro-2-(4-Chlorophenyl)-1-Methyl-1H-Indol-3-yl]-2-Propenoate

A mixture of 5-chloro-2-(4-chlorophenyl)-1-methyl-1H-indole-3-carboxaldehyde (Description 18, 347 mg, 1.1 mmol) and ethyl (triphenylphosphoranylidiene) acetate (500 mg) in chloroform (15 mL) was heated under reflux for 24 h. Further ethyl (triphenylphosphoranylidiene) acetate (870 mg) was added and the mixture was heated under reflux for 24 h. A third portion of ethyl (triphenylphosphoranylidiene)acetate (980 mg) was added and the mixture was heated under reflux for 24 h. The mixture was cooled and the solvent was evaporated under reduced pressure. The residue was purified by flash colunm chromatography on silica gel, eluting with hexane-EtOAc (85:15) to give the title compound as a yellow solid (298 mg, 70%). ¹H NMR (CDCl₃) δ7.95 (1H, br s), 7.60 (1H, d, J 16 Hz), 7.56-7.50 (2H, m), 7.37-7.29 (4H, m), 6.40 (1H, d, J 16 Hz), 4.22 (2H, q, J 7 Hz), 3.61 (3H, s), and 1.31 (3H, t, J 7 Hz).

### DESCRIPTION 20

### (Z)-Methyl [5-Chloro-2-(4-Chlorophenyl)- 1-Methyl-1H-Indol-3-yl]-2-Propenoate

Potassium hexamethyldisilazide (0.5M in toluene, 3.0 mL) was added over 2 min. to a stirred, cooled (−78 ° C.) solution of 18-crown-6 (2.0 g) in tetrahydrofuran (20 mL), followed by methyl [bis(2,2,2-trifluoroethoxy)-phosphinyl]acetate (320 μL). 5-Chloro-2-(4-chlorophenyl)-1-methyl-1H-indole-3-carboxaldehyde (Description 18, 300 mg, 1.0 mmol) in tetrahydrofuran (3 mL) was added dropwise over 5 min., and the mixture was allowed to warm to room temperature and stirred for 16 h. Aqueous ammonium chloride (saturated, 20 mL) and water (5 mL) were added and the mixture was extracted with ethyl acetate (2×20 mL). The combined organic fractions were dried (MgSO₄) and the solvent was evaporated under reduced pressure. The residue was purified by flash column chromatography on silica gel, eluting with hexane-EtOAc (90:10) to give the title compound (50 mg, 14%). ¹H NMR (CDCl₃) δ7.48 (2H, d, J 8.4 Hz), 7.37-7.32 (3H, m), 7.27 (1H, d, J 8.6 Hz), 7.23 (1H, dd, J 8.6, 1.8 Hz), 6.75 (1H, d, J 12 Hz), 5.91 (1H, d, J 12 Hz), 3.67 (3H, s), and 3.64 (3H, s).

### DESCRIPTION 21

### 3-(2,2-Dibromoethenyl)-5-Chloro-2-(4-Chlorophenyl)-1-Methyl-1H-Indole

A solution of triphenylphosphine (713 mg) in dichloromethane was added dropwise to stirred, cooled (−20 ° C.) solution of tetrabromoethane (902 mg) in dichloromethane (30 mL). The mixture was stirred at −20 ° C. for 15 min., cooled to −60 ° C. and a solution of 5-chloro-2-(4-chlorophenyl)-1-methyl-1H-indole-3-carboxaldehyde (Description 18, 412 mg, 1.4 mmol) and triethylamine (190 μL) in dichloromethane (15 mL) was added over 10 min. The mixture was allowed to warm to room temperature and stirred for 16 h. The solvent was evaporated under reduced pressure and the residue was triturated with hexane—CH₂Cl₂. The mixture was filtered and the solvent was evaporated under reduced pressure. The residue was purified by flash column chromatography on silica gel, eluting with hexane-EtOAc (90:10) to give the title compound (474 mg, 76%). ¹H NMR (CDCl₃) δ7.66 (1H, br s), 7.50 (2H, d, J 8 Hz), 7.35-7.31 (3H, m), 7.28 (1H, d, J 8.7 Hz), 7.24 (1H, dd, J 8.7, 1.9 Hz), and 3.66 (3H, s).

### DESCRIPTION 22

### Methyl [5-Chloro-2-(4-Chlorophenyl)-1-Methyl-1H-Indol-3-yl]-2-Propynoate

n-Butyl lithium (1.6M in hexanes, 1.24 mL) was added dropwise to a stirred, cooled (−78 ° C.) solution of 3-(2,2-dibromoethenyl)-5-chloro-2-(4-chlorophenyl)-1-methyl-1H-indole (Description 21, 435 mg, 0.94 mmol) in tetrahydrofuran (4 mL). Methyl cyanoformate (500 μL) was added and the mixture was allowed to warm to room temperature. Water (10 mL) was added and the mixture was extracted with ethyl acetate (2×10 mL). The combined organic fractions were dried (MgSO₄) and the solvent was evaporated under reduced pressure. The residue was purified by flash column chromatography on silica gel, eluting with hexane/EtOAc (75:25) to give the title compound (32 mg, 9%). ¹H NMR (CDCl₃) δ7.8 (1H, br s), 7.56-7.49 (4H, m), 7.29 (2H, br s), 3.79 (3H, s), and 3.72 (3H, s).

### DESCRIPTION 23

### 5-Chloro-2-(4-Chlorophenyl)-1-Methyl-1H-Indole-3-Propanal

Diisobutylaluminium hydride (1M in hexane, 0.5 nmL) was added to a stirred, cooled (−78 ° C.) solution of methyl 5-chloro-2-(4-chlorophenyl)-1-methyl-1H-indole-3-propanoate (Description 10, 181 mg, 0.5 mmol) in dichloromethane (20 mL) and hexane (80 mL). The mixture was stirred at −78° C. for 4 h., then methanol (2 mL) was added and the mixture was allowed to warm to room temperature. The mixture was poured into hydrochloric acid (1M) and extracted with ethyl acetate (3×100 mL). The combined organic fractions were dried (MgSO₄) and the solvent was evaporated under reduced pressure to give the title compound as a colorless oil (160 mg, 96%). ¹H NMR (360MHz, CDCl₃) δ9.68 (1H, t, J 1.5 Hz), 7.46 (1H, d, J 1.5 Hz), 7.43-7.39 (2H, m), 7.25-7.11 (4H, m), 3.46 (3H, s), 2.92-2.87 (2H, m), and 2.56-2.61 (2H, m).

### DESCRIPTION 24

### 1-Acetyl-4-Cyclohexyl-4-Piperidinol

A solution of 1-acetyl-4-piperidinone (2.46 mL, 20 mmol) in tetrahydrofuran (50 mL) was added dropwise to a stirred, cooled (0° C.) solution of of cyclohexylmagnesium bromide (2M in diethyl ether, 10 mL, 20 mmol). The mixture was allowed to warm to room temperature, aqueous ammonium chloride (saturated, 50 mL) was added and the mixture was extracted with ethyl acetate (3×100 mL). The combined organic fractions were dried (MgSO₄) and the solvent was evaporated under reduced pressure. The residue was purified by flash column chromatography on silica gel, eluting with ethyl acetate/methanol (100:0 increasing to 90:10) to give the title compound as a colorless solid (602 mg, 15%). ¹H NMR (360MHz, CDCl₃) δ1.02-1.36 (5H, m), 1.64-1.84 (6H, m), 2.08 (3H, s), 2.44-2.50 (1H, m), 2.84-2.94 (2H, m), 3.38-3.46 (2H, m), 2.56-2.64 (2H, m), and 4.42-4.50 (1H, m). m/z (ES⁺) 226 (M+1).

### DESCRIPTION 25

### 4-Cyclohexyl-4-Piperidinol

Aqueous potassium hydroxide (40%, 15 mL) was added to a solution 1-acetyl-4-cyclohexyl-4-piperidinol (Description 24, 600 mg, 2.7 mmol) in methanol (25 mL) and the mixture was heated to 60° C. for 16 h. The mixture was cooled and extracted with ethyl acetate (3×50 mL). The combined organic fractions were dried (MgSO₄) and the solvent was evaporated under reduced pressure to give the title compound as a colorless solid (492 mg, 99%). ¹H NMR (360MHz, CDCl₃) δ0.96-1.24 (6H, m), 1.51-1.69 (5H, m), 1.79-1.82 (4H, m), 1.96 (1H, s), 2.50-2.80 (2H, br s), and 2.82-3.02 (4H, m). m/z (ES⁺) 184 (M+1).

### DESCRIPTION 26

### 4-(Dimethylamino)-1-(Phenylmethyl)-4-Piperidinecarbonitrile

A solution of 1-(phenylmethyl)-4-piperidone (9.46 g, 50 mmol) in ethanol (20 mL) was added slowly to a stirred solution of potassium cyanide (3.58 g, 55 mmol) and dimethylamine hydrochloride (4.89 g, 60 mmol) in water (60 mL). The mixture was stirred at room temperature for 68 h., then water (100 mL) was added. The solid was collected, suspended in saturated aqueous sodium hydrogen carbonate (100 mL) and water (50 mL) and extracted with dichloromethane (3×100 mL). The combined organic fractions were dried (MgSO₄) and the solvent was evaporated under reduced pressure to give the the title compound as a cream solid (11.69 g, 96%). ¹H NMR (360 MHz, CDCl₃) δ7.34-7.22 (5H, m), 3.53 (2H, s), 2.85 (2H, m), 2.33 (6H, s), 2.31 (2H, mn), 2.11 (2H, mn), and 1.74 (2H, m). m/z (ES⁺) 244 (M+1).

### DESCRIPTION 27

### 4-(Dimethylamino)-1,4-bis(Phenylmethyl)Piperidine

A solution of 4-(dimethylamino)-1-(phenylmethyl)-4-piperidine carbonitrile (Description 26, 4.86 g, 20 mmol) in ether (75 mL) was added to benzylmagnesium chloride (1.0M in ether, 100 mL, 100 mmol) and the mixture was heated under reflux for 6 h. The mixture was cooled in ice and hydrochloric acid (1M, 100 mL) was added slowly. The layers were separated and the aqueous layer was extracted with hydrochloric acid (1M, 2×100 mL). The combined aqueous layers were washed with ether (100 mL) then adjusted to pH 10.0 with aqueous sodium hydroxide (4M). The mixture was extracted with ether (3×200 mL) and the combined organic fractions were evaporated under reduced pressure. Saturated aqueous sodium hydrogen carbonate (100 mL) and water (20 mL) were added and the mixture was extracted with dichloromethane (3×100 mL). The combined organic fractions were dried (MgSO₄) and the solvent was evaporated under reduced pressure. The residue was recrystallized from ethanol-water (2:1, 75 mL) to give the the title compound as a colorless solid (5.15 g, 84%). ¹H NMR (360MHz, CDCl₃) δ7.28-7.09 (1OH, m), 3.44 (2H, s), 2.64 (2H, s), 2.49 (2H, m), 2.36 (2H, m), 2.30 (6H, s), 1.72 (2H, m), and 1.44 (2H, m). m/z (ES⁺) 309 (M+1).

### DESCRIPTION 28

### 4-(Dimethylamino)-4-(Phenylmethyl)Piperidine

A suspension of palladium on carbon (10%, 2 g) was added to a solution of 4-(dimethylamino)-1,4-bis(phenylmethyl)piperidine (Description 27, 4.62 g, 15 mmol) and formic acid (90%, 1.4 mL) in methanol (100 mL). Ammonium formate (4.73 g, 75 mmol) was added and the mixture was stirred at room temperature for 20 h. The mixture was filtered, washing with methanol, and the solvent was evaporated under reduced pressure. Ether (40 mL) was added and the mixture was extracted with hydrochloric acid (1M, 3×40 mL). The combined aqueous layers were washed with ether (40 mL), adjusted to pH 12.0 with aqueous sodium hydroxide (4M) and extracted with dichloromethane (3×40 mL). The combined organic fractions were dried (MgSO₄) and the solvent was evaporated under reduced pressure. The residue was triturated with hexane (20 mL) and the solid was collected and dried in vacuo to give the the title compound as a colorless solid (2.20 g, 67%). ¹H NMR (360 MHz, CDCl₃) δ7.24 (2H, t, J 7.0 Hz), 7.17 (1H, t, J 7.0 Hz), 7.11 (2H, d, J 7.0 Hz), 2.96 (2H, m), 2.67 (2H, m), 2.63 (2H, s), 2.31 (6H, s), 1.73 (2H, m), 1.45 (1H, br s) and 1.28 (2H, m). m/z (ES⁺) 219 (M+1).

### DESCRIPTION 29

### 1,1-Dimethylethyl 4-(Trifluoromethanesulfonyloxy)-1,2,3,6-Tetrahydropyridine-1-Carboxylate

A solution of 1,1-dimethylethyl 4-oxo-1-piperidinecarboxylate (1.0 g) in tetrahydrofuran (10 mL) was added dropwise to a stirred, cooled (−78 ° C.) solution of lithium diisopropylamide [freshly prepared from diisopropylamine (555 mg) and n-butyllithium (1.6M in hexane, 3.5 mL)] in tetrahydrofuran (40 mL) and the mixture was stirred at −78 ° C. for 20 min. A solution of N-phenylbis(trifluoromethanesulfonimide) (1.96 g) in tetrahydrofuran (10 mL) was added and the solution was allowed to warm to room temperature and stirred for 1 h. The solvent was evaporated under reduced pressure and the residue was partitioned between ethyl acetate and water. The organic layer was dried (MgSO₄) and the solvent was evaporated under reduced pressure. The residue was purified by chromatography on alumina (ICN GIII) eluting with EtOAc/Hexane (10:90) to give the title compound as a colorless solid (1.31 g). ¹H NMR (CDCl₃) δ1.47 (9H, s), 2.44 (2H, m), 3.63 (2H, t, J 7.0 Hz), 4.04 (2H, m), and 5.76 (1H, br s).

### DESCRIPTION 30

### 4-(2-Methoxyphenyl)Piperidine Hydrochloride

Tetrakis(triphenylphosphine)palladium (0) (100 mg) was added to a degassed mixture of 1,1-dimethylethyl 4-(trifluoromethanesulfonyloxy)-1,2,3,6-tetrahydropyridine-1-carboxylate (Description 29, 810 mg), (2-methoxyphenyl)boronic acid (519 mg), lithium chloride (405 mg) and aqueous sodium carbonate (2N, 3.5 mL) in 1,2-dimethoxyethane (20 mL). The mixture was heated under reflux for 3 h., cooled to room temperature and the solvent was evaporated under reduced pressure. The residue was partitioned between ethyl acetate and aqueous sodium carbonate (2M), the organic layer was separated, dried (MgSO₄) and the solvent was evaporated under reduced pressure. The residue was dissolved in ethanol, palladium on carbon (10%) was added and the mixture was shaken under hydrogen (50 psi.) for 3 h. The mixture was filtered through Hyflo™ and the solvent was evaporated under reduced pressure. The residue was purified by MPLC on silica gel, eluting with EtOAc/Hexane (5:95) and the residue was deprotected with ethanolic hydrogen chloride to give the title compound as a colorless solid (340 mg). ¹H NMR (DMSO-d₆) δ1.54-1.59 (2H, m), 1.77-1.81 (2H, m), 2.77 (2H, td, J 11.0, 1.0 Hz), 3.07 (1H, td, J 11.0, 1.0 Hz), 3.11 (2H, br m), 3.82 (3H, s), 6.85 (1H, d, J 6.0 Hz), 6.93 (1H, t, J 6.0 Hz), 7.15-7.21 (2H, m).

### DESCRIPTION 31

### 1,1-Dimethylethyl 4-Aminomethyl-4-Phenylpiperidine-1-Carboxylate

Di-t-butyldicarbonate (13.10 g, 60 mmol) in 1,4-dioxane (50 mL) was added to a stirred mixture of 4-cyano-4-phenylpiperidine hydrochloride (11.14 g, 50 mmol) and sodium carbonate (13.25 g, 125 mmol) in water (150 mL) and the mixture was stirred at room temperature for 6 h. Water (150 mL) was added and the mixture was extracted with ethyl acetate (3×150 mL). The combined organic fractions were washed with aqueous citric acid (10%, 2×100 mL), aqueous sodium hydrogen carbonate (saturated, 100 mL) and brine (100 mL), dried (MgSO₄) and the solvent was evaporated under reduced pressure. The residue was dissolved in ethanol-acetic acid (95:5, 200 mL), platinum oxide (1 g) was added and the mixture was shaken under hydrogen (50 psi) for 22 h., adding further platinum oxide (1 g) after 4 h. The mixture was filtered through Hyflo™, further ethanol (85 mL), acetic acid (15 mL) and platinum oxide (1 g) were added and the mixture was shaken under hydrogen (50 psi) for 46 h., adding further platinum oxide (1 g) after 22 h. The mixture was filtered through Hyflo™ and the solvent was evaporated under reduced pressure. Aqueous ammonia (saturated, 200 mL) was added and the mixture was extracted with ethyl acetate (3×200 mL). The combined organic fractions were dried (MgSO₄) and the solvent was evaporated under reduced pressure to give the crude title compound as an orange oil (15.92 g). ¹H NMR (250 MHz, CDCl₃) δ7.41-7.21 (5H, m), 3.74 (2H, m), 3.05 (2H, m), 2.75 (2H, s), 2.19 (2H, m), 1.80 (2H, br s), 1.69 (2H, m), and 1.43 (9H, s).

### DESCRIPTION 32

### N-[1-(1,1-Dimethylethoxycarbonyl)-4-Phenylpiperidine-4-Methyl]Acetamide

Acetic anhydride (2.59 mL, 2.81 g, 27.5 mmol) was added dropwise to a stirred, cooled (0° C.) solution of 1,1-dimethylethyl 4-aminomethyl-4-phenylpiperidine-1-carboxylate (Description 31, 7.96 g) and pyridine (3.03 mL, 2.97 g, 37.5 mmol) in dichloromethane (100 mL) and the mixture was stirred at room temperature for 18 h. The solvent was evaporated under reduced pressure, aqueous sodium hydrogen carbonate (saturated, 100 mL) was added and the mixture was extracted with ethyl acetate (3×100 mL). The combined organic fractions were washed with aqueous citric acid (10%, 2×100 mL), aqueous sodium hydrogen carbonate (saturated, 100 mL) and brine (100 mL), dried (MgSO₄) and the solvent was evaporated under reduced pressure. The residue was purified by flash column chromatography on silica gel, eluting with ethyl acetatelhexane (50:50 increasing to 100:0) to give the title compound as an off-white solid (5.96 g, 72% from 4-cyano-4-phenylpiperidine hydrochloride). ¹H NMR (250 MHz, CDCl₃) δ7.45-7.26 (5H, m), 5.00 (1H, br m), 3.67 (2H, m), 3.45 (2H, br m), 3.21 (2H, m), 2.08 (2H, m), 1.88 (3H, s), 1.78 (2H, m), and 1.43 (9H, s).

### DESCRIPTION 33

### N-(4-Phenylpiperidine-4-Methyl)Acetamide Hydrochloride

Methanolic hydrogen chloride (4M, 40 mL) was added to a stirred, cooled (0° C.) suspension of N-[1-(1,1-dimethylethoxycarbonyl)-4-phenylpiperidine-4-methyl]acetamide (Description 32, 5.90 g, 17.8 mmol) in methanol (40 mL) and the mixture was stirred at room temperature for 20 h. The solvent was evaporated under reduced pressure to give the title compound as a tan foam (4.67 g, 98%). ¹H NMR (250 MHz, DMSO-d₆) δ9.09 (1H, br s), 8.88 (1H, br s), 7.73 (1H, t, J 6.3 Hz), 7.43-7.24 (5H, m), 3.18 (4H, m), 2.70 (2H, m), 2.21 (2H, m), 1.97 (2H, m), and 1.76 (3H, s).

### DESCRIPTION 34

### 1-(1,1-Dimethylethoxycarbonyl)Piperidine-4-Carboxylic Acid

Di-t-butyldicarbonate (23.42 g, 107.3 mmol) in dichloromethane (100 mL) was added slowly to a mixture of 4-piperidinecarboxylic acid (12.60 g, 97.6 mmol) and triethylamine (13.60 mL, 9.87 g, 97.6 mmol) in dichloromethane (50 mL) and the mixture was stirred at room temperature for 18 h. N,N-Dimethylethylenediamine (3.46 mL, 2.87 g, 32.5 mmol) was added and the mixture was stirred at room temperature for 30 min. Dichloromethane (100 mL) was added and the mixture was washed with aqueous citric acid (10%, 2×200 mL), dried (MgSO₄) and the solvent was evaporated under reduced pressure to give the title compound as a colorless solid (21.05 g, 94%). 1H NMR (250MHz, CDCl₃) δ4.02 (2H, m), 2.86 (2H, m), 2.49 (1H, m), 1.91 (2H, m), 1.64 (2H, m), and 1.46 (9H, s).

### DESCRIPTION 35

### N-Phenyl-1-(1,1-Dimethylethoxycarbonyl)Piperidine-4-Carboxamide

Triethylamine (10.04 mL, 7.28 g, 72 mmol) was added to a stirred, cooled (0° C.) mixture of 1-(1,1-dimethylethoxycarbonyl)piperidine-1-carboxylic acid (Description 34, 6.87 g, 30 mmol), aniline (2.73 mL, 2.79 g, 30 mmol) and bis(2-oxo-3-oxazolidinyl)phosphinic chloride (9.16 g, 36 mmol) in dichloromethane (50 mL) and the mixture was stirred at room temperature for 18 h. The solvent was evaporated under reduced pressure, water (50 mL) was added and the mixture was extracted with ethyl acetate (4×50 mL). The combined organic fractions were washed with aqueous citric acid (10%, 2×50 mL), aqueous sodium hydrogen carbonate (saturated, 2×50 mL) and brine (50 mL), dried (MgSO₄) and the solvent was evaporated under reduced pressure. The residue was purified by flash column chromatography on silica gel, eluting with EtOAc/Hexane (40:60) to give the title compound as a colorless foam (7.23 g, 79%). ¹H NMR (250 MHz, CDCl₃) δ7.51 (2H, d, J 7.6 Hz), 7.32 (2H, t, J 7.6 Hz), 7.26 (1H, br s), 7.11 (1H, t, J 7.6 Hz), 4.19 (2H, m), 2.78 (2H, m), 2.38 (1H, m), 1.90 (2H, m), 1.77 (2H, m), and 1.47 (9H, s). m/z (ES⁺) 305 (M+1).

### DESCRIPTION 36

### N-Phenyl-1-(1,1-Dimethylethoxycarbonyl)Piperidine-4-Methylamine

Borane-tetrahydrofuran complex (1.0 M in tetrahydrofuran, 57 mL, 57 mmol) was added to a stirred, cooled (0° C.) solution of N-phenyl-1-(1,1-dimethylethoxycarbonyl)piperidine-4-carboxamide (Description 35, 5.78 g, 19 mmol) in tetrahydrofuran (95 mL) and the mixture was stirred at room temperature for 18 h. Methanol (10 mL) was added and the solvent was evaporated under reduced pressure. Potassium carbonate (13.13 g, 95 mmol) and methanol (150 mL) were added and the mixture was heated under reflux for 1 h. The mixture was cooled and the solvent was evaporated under reduced pressure. Water (100 mL) was added and the mixture was extracted with dichloromethane (3×100 mL). The combined organic fractions were dried (MgSO₄) and the solvent was evaporated under reduced pressure. The residue was purified by flash column chromatography on silica gel, eluting with EtOAc/Hexane (20:80) to give the title compound as a colorless solid (5.08 g, 92%). ¹H NMR (250 MHz, CDCl₃) δ7.18 (2H, t, J 7.6 Hz), 6.69 (1H, t, J 7.6 Hz), 6.59 (2H, d, J 7.6 Hz), 4.14 (2H, m), 3.73 (1H, br s), 3.03 (2H, d, J 6.2 Hz), 2.69 (2H, m), 1.79-1.55 (3H, m), 1.46 (9H, s), and 1.20 (2H, m).

### DESCRIPTION 37

### N-[1-(1,1-Dimethylethoxycarbonyl)Piperidine-4-Methyl]-N-Phenylmethanesulfonamide

Methanesulfonyl chloride (0.77 mL, 1.13 g, 9.9 mmol) was added dropwise to a stirred, cooled (0° C.) solution of N-phenyl-1-(1,1-dimethylethoxycarbonyl)piperidine-4-methylamine (Description 36, 2.61 g, 9 mmol) and pyridine (1.09 mL, 1.07 g, 13.5 mmol) in dichloromethane (50 mL) and the mixture was stirred at room temperature for 16 h. The mixture was cooled in ice and 4-dimethylaminopyridine (220 mg, 1.8 mmol), pyridine (1.09 mL, 1.07 g, 13.5 mmol) and methanesulfonyl chloride (0.77 mL, 1.13 g, 9.9 mmol) were added. The mixture was stirred at room temperature for 24 h. The solvent was evaporated under reduced pressure, aqueous sodium hydrogen carbonate (saturated, 50 mL) was added and the mixture was extracted with ethyl acetate (3×50 mL). The combined organic fractions were washed with aqueous citric acid (10%, 2×50 mL), aqueous sodium hydrogen carbonate (saturated, 50 mL) and brine (50 mL), dried (MgSO₄) and the solvent was evaporated under reduced pressure. The residue was recrystallized from ethyl acetate/hexane (2:1, 30 mL) to give the title compound as a colorless solid (2.96 g, 89%). ¹H NMR (250 MHz, CDCl₃) δ7.46-7.31 (5H, m), 4.06 (2H, m), 3.56 (2H, d, J 7.2 Hz), 2.85 (3H, s), 2.60 (2H, m), 1.71 (2H, m), 1.57 (1H, m), 1.43 (9H, s), and 1.05 (2H, m).

### DESCRIPTION 38

### N-Phenyl-N-(Piperidine-4-Methyl)Methanesulfonamide Hydrochloride

Methanolic hydrogen chloride (4M, 20 mL) was added to a stirred, cooled (0° C.) suspension of N-[1-(1,1-dimethylethoxycarbonyl)piperidine-4-methyl]-N-phenylmethanesulfonamide (Description 37, 2.84 g, 7.7 mmol) in methanol (10 mL) and the mixture was stirred at room temperature for 1 h. The solvent was evaporated under reduced pressure to give the title compound as a tan foam (2.34 g, 100%). ¹H NMR (250 MHz, DMSO-d₆) δ8.90 (2H, br m), 7.45-7.33 (5H, m), 3.53 (2H, d, J 7.0 Hz), 3.19 (2H, m), 2.96 (3H, s), 2.73 (2H, m), 1.80 (2H, m), 1.54 (1H, m), and 1.36 (2H, m).

### DESCRIPTION 39

### 5-Chloro-1H-Indole-3-Propanoic Acid

Acrylic acid (34 mL, 496 mmol) was added to a solution of 5-chloro-1H-indole (25 g, 165 mmol) in acetic acid (50 mL) and acetic anhydride (50 mL) and the mixture was stirred at room temperature for 1 week. Aqueous sodium hydroxide (4N, 100 mL) was added and the mixture was washed with ethyl acetate. The aqueous fraction was acidified to pH 1 with hydrochloric acid (5M) and extracted with ethyl acetate. The combined organic fractions were dried (MgSO₄) and the solvent was evaporated under reduced pressure. The residue was purified by flash column chromatography on silica gel, eluting with ether, to give the title compound as a colorless solid (17.2 g, 47%). ¹H NMR (360 MHz, CDCl₃) δ7.98 (1H, br s), 7.56 (1H, d, J 2.0 Hz), 7.26 (1H, d, J 8.4 Hz), 7.15 (1H, dd, J 8.4, 2.0 Hz), 7.05 (1H, d, J 2.3 Hz), 3.07 (2H, t, J 7.5 Hz), and 2.75 (2H, t, J 7.5 Hz.

### DESCRIPTION 40

### Methyl 5-Chloro-1-Methyl-1H-Indole-3-Propanoate

Sodium hydride (60% suspension in mineral oil, 0.90 g, 22.3 mmol) was added in portions to a stirred, cooled (0° C.) solution of 5-chloro-1H-indole-3-propanoic acid (Description 39, 2 g, 8.9 mmol) in dimethylformamide (30 mL) and the mixture was stirred at rooom temperature for 1 h. Iodomethane (2.8 mL, 44.5 mmol) was added and the mixture was stirred at room temperature for 30 min. Water (100 mL) was added and the mixture was extracted with ethyl acetate. The combined organicfractions were washed with brine, dried (MgSO₄) and the solvent was evaporated under reduced pressure. The residue was was purified by flash column chromatography on silica gel, eluting with EtOAc/hexane (20:80) to give the title compound as a colourless oil (2.1 g, 94%). ¹H NMR (360 MHz, CDCl₃) δ7.56 (1H, d, J 1.7 Hz), 7.20-7.13 (2H, m), 6.88 (1H, s), 3.72 (3H, s), 3.68 (3H, s), 3.03 (2H, t, J 7.6 Hz), and 2.67 (2H, t, J 7.6 Hz).

### DESCRIPTION 41

### Methyl 2-Bromo-5-Chloro-1-Methyl-1H-Indole-3-Propanoate

A solution of bromotrimethylsilane (1.6 mL, 11.9 mmol) in dimethylsulfoxide (10 mL) was added dropwise to a stirred solution of methyl 5-chloro-1-methyl-1H-indole-3-propanoate (Description 40, 2 g, 7.95 mmol) in dimethylsulfoxide (20 mL) and the mixture was stirred at room temperature for 18 h. Aqueous sodium carbonate (saturated) was added and the mixture was extracted with ethyl acetate. The combined organic fractions were washed with brine, dried (MgSO₄) and the solvent was evaporated under reduced pressure. The residue was was purified by flash column chromatography on silica gel, eluting with EtOAc/hexane (25:75) to give the title compound as a pale yellow solid (1.4 g, 53%). IH NMR (360 MHz, CDCl₃) δ7.50 (1H, d, J 1.7 Hz), 7.17-7.15 (2H, m), 3.72 (3H, s), 3.68 (3H, s), 3.04 (2H, m), and 2.61 (2H, m).

### DESCRIPTION 42

### Methyl 5-Chloro-2-(4-Fluorophenyl)- 1-Methyl-1H-Indole-3-Propanoate

A mixture of methyl 2-bromo-5-chloro-1-methyl-1H-indole-3-propanoate (Description 41, 200 mg, 0.6 mmol), 4-fluorobenzene boronic acid (170 mg, 1.7 mmol) and potassium carbonate (100 mg) in dimethoxyethane (10 mL) was degassed. Tetrakis(triphenylphosphine) palladium (0) (35 mg, 0.03 mmol) was added and the mixture was degassed and stirred at 75° C. for 48 h. The mixture was cooled, water was added and the mixture was extracted with ethyl acetate. The combined organic fractions were washed with brine, dried (MgSO₄) and the solvent was evaporated under reduced pressure. The residue was was purified by flash column chromatography on silica gel, eluting with EtOAc/hexane (15:85) to give the title compound as a pale yellow oil (190 mg, 92%). ¹H NMR (360 MHz, CDCl3) δ7.57 (1H, d, J 1.7 Hz), 7.36-7.32 (2H, m), 7.22-7.12 (4H, m), 3.67 (3H, s), 3.52 (3H, s), 2.96 (2H, m), and 2.53 (2H, m).

### DESCRIPTION 43

### Methyl 5-Chloro-1-Methyl-2-(2-Pyridinyl)-1H-Indole-3-Propanoate

Prepared from methyl 2-bromo-5-chloro-1-methyl-1H-indole-3-propanoate (Description 41) and 2-(tributylstannyl)pyridine, according to the method of Description 14. ¹H NMR (360 MHz, CDCl₃) δ8.78 (1H, d, J 4.1 Hz), 7.83 (1H, m), 7.60 (1H, d, J 1.9 Hz), 7.50 (1H, d, J 7.7 Hz), 7.32 (1H, m), 7.27 (1H, d, J 8.7 Hz), 7.21 (1H, dd, J 8.7, 1.9 Hz), 3.71 (3H, s), 3.62 (3H, s), 3.11 (2H, t, J 8.0 Hz), and 2.61 (2H, t, J 8.0 Hz). m/z (ES⁺) 329, 331 (M+1).

### DESCRIPTION 44

### Methyl 5-Chloro-1-Methyl-2-(3-Pyridinyl)-1H-Indole-3-Propanoate

Prepared from methyl 2-bromo-5-chloro-1-methyl-1-H-indole-3-propanoate (Description 41) and 3-(tributylstannyl)pyridine, according to the method of Description 14. ¹H NMR (360 MHz, CDCl₃) 8 8.71 (1H, dd, J 4.9, 1.8 Hz), 8.65 (1H, d, J 1.8 Hz), 7.73 (1H, dt, Jd 7.8, Jt 1.8 Hz), 7.60 (1H, d, J 1.8 Hz), 7.46 (1H, dd, J 7.8, 4.9 Hz), 7.26 (1H, d, J 8.6 Hz), 7.23 (1H, dd, J 8.6, 1.8 Hz), 3.60 (3H, s), 3.56 (3H, s), 2.99 (2H, t, J 7.9 Hz), and 2.56 (2H, t, J 7.9 Hz). m/z (ES⁺) 329, 331 (M+1).

### DESCRIPTION 45

### 6-Methyl-3-Pyridinyl Trifluoromethanesulfonate

Trifluoromethanesulfonic anhydride (11.6 mL, 19.5 g, 69 mmol) was added slowly to a stirred, cooled (0° C.) solution of 6-methyl-3-pyridinol (5 g, 46 mmol) and triethylamine (32.0 mL, 23.3 mmol) in tetrahydrofuran (100 mL) and the mixture was stirred at room temperature for 18 h. The mixture was poured into water and extracted with ethyl acetate. The combined organic fractions were washed with saturated aqueous sodium hydrogen carbonate solution and brine, dried (MgSO₄) and the solvent was evaporated under reduced pressure. The residue was purified by flash column chromatography on silica gel, eluting with hexanes/EtOAc (65:35), to give the title compound as a yellow oil (4 g, 35%). ¹H NMR (360 MHz, DMSO-d₆) δ8.64 (1H, d, J 3.0 Hz), 7.93 (1H, dd, J 8.7, 3.0 Hz), 7.48 (1H, d, J 8.7 Hz), and 2.51 (3H, s). m/z (ES⁺) 242 (M+1).

### DESCRIPTION 46

### 2-Methyl-5-(Trimethylstannyl)Pyridine

A mixture of 6-methyl-3-pyridinyl trifluoromethanesulfonate (Description 45, 3 g, 12.4 mmol), lithium carbonate (0.92 g, 12.4 mmol), lithium chloride (3.14 g, 74 mmol) and hexamethylditin (5 g, 15.3 mmol) in tetrahydrofuran was degassed with bubbling nitrogen. Tetrakis(triphenylphosphine)palladium (0) (0.72 mg, 0.6 mmol) was added and the mixture was degassed with bubbling nitrogen, then heated under reflux for 72 h. The mixture was cooled and a solution of potassium fluoride (10 g) in water (50 mnL) was added. The mixture was stirred at room temperature for 1 h., filtered through a glass fibre filter and extracted with ethyl acetate. The combined organic fractions were washed with brine, dried (MgSO₄) and the solvent was evaporated under reduced pressure. The residue was extracted with hexane and the solvent was evaporated under reduced pressure to give the title compound as a colorless oil (2 g, 53%). ¹H NMR (360 MHz, CDCl₃) δ7.65 (1H, dd, J 7.4, 1.8 Hz), 7.33 (1H, d, J 1.8 Hz), 7.12 (1H, d, J 7.4 Hz), 2.53 (3H, s), and 0.31 (9H, s). m/z (ES⁺) 254, 256, 258 (M+1).

### DESCRIPTION 47

### 5-(Trifluoromethyl)-2-(Trimethylstannyl)Pyridine

Prepared from 2-bromo-5-(trifluoromethyl)pyridine according to the method of Description 46. ¹H NMR (360 MHz, CDCl₃) δ8.99 (1H, d, J 2.3 Hz), 7.73 (1H, dd, J 7.8, 2.3 Hz), 7.60 (1H, d, J 7.8 Hz), and 0.38 (9H, s).

### DESCRIPTION 48

### Methyl 5-Chloro-1-Methyl-2-(6-Methyl-3-Pyridinyl)-1H-Indole-3-Propanoate

Prepared from methyl 2-bromo-5-chloro-1-methyl-1H-indole-3-propanoate (Description 41) and 2-methyl-5-(trimethylstannyl)pyridine (Description 46), according to the method of Description 14. ¹H NMR (400 MHz, CDCl₃) δ8.52 (1H, d, J 1.8 Hz), 7.60 (1H, dd, J 7.9, 1.8 Hz), 7.56 (1H, d, J 1.5 Hz), 7.31 (1H, d, J 7.9 Hz), 7.22 (2H, m), 3.60 (3H, s), 3.55 (3H, s), 2.95 (2H, m), 2.65 (3H, s), and 2.55 (2H, m). m/z (ES⁺) 343, 345 (M+1).

### DESCRIPTION 49

### Methyl 5-Chloro-1-Methyl-2-[5-(Trifluoromethyl)-2-Pyridinyl]-1H-Indole-3-Propanoate

Prepared from methyl 2-bromo-5-chloro-1-methyl-1H-indole-3-propanoate (Description 41) and 5-(trifluoromethyl)-2-(trimethylstannyl) pyridine (Description 47), according to the method of Description 14. ¹H NMR (360 MHz, CDCl₃) δ9.04 (1H, d, J 2.3 Hz), 8.07 (1H, dd, J 8.2, 2.3 Hz), 7.68 (1H, d, J 8.2 Hz), 7.62 (1H, d, J 1.8 Hz), 7.30 (1H, d, J 8.7 Hz), 7.25 (1H, dd, J 8.7, 1.8 Hz), 3.74 (3H, s), 3.62 (3H, s), 3.33 (2H, t, J 8.0 Hz), and 2.65 (2H, t, J 8.0 Hz). m/z (ES⁺) 397, 399 (M+1).

### DESCRIPTION 50

### 5-Chloro-2-(4-Chlorophenyl)-1-Methyl-1H-Indole-3-Propanenitrile

Oxalyl chloride (1 mL, 1.46 g, 11.4 mmol) and dimethylformamide (3 drops) were added to a solution of 5-chloro-2-(4-chlorophenyl)-1-methyl-1H-indole-3-propanoic acid (Description 11, 2.0 g, 5.7 mmol) in dichloromethane (30 mL) and the mixture was stirred at room temperature for 1 h. The solvent was evaporated under reduced pressure and toluene (10 mL) was added. The solvent was evaporated under reduced pressure and toluene (10 mL) was added. The solvent was evaporated under reduced pressure and the residue was dissolved in tetrahydrofuran (20 mL). Aqueous ammonia (saturated, 10 mL) was added and the mixture was stirred at room temperature for 18 h. Water (100 mL) was added and the mixture was extracted with ethyl acetate (2×100 mL). The combined organic fractions were washed with brine (50 mL), dried (MgSO₄) and the solvent was evaporated under reduced pressure. The residue was dissolved in phosphorus oxychloride (25 mL) and the mixture was heated under reflux for 10 min. The mixture was cooled and poured onto ice. The mixture was basified with aqueous sodium hydroxide (4M) and extracted with ethyl acetate (2×100 mL). The combined organic fractions were washed with brine (50 mL), dried (MgSO₄) and the solvent was evaporated under reduced pressure. The residue was purified by flash column chromatography on silica gel, eluting with isohexane/EtOAc (80:20), to give the title compound as a yellow solid (0.89 g, 47%). (360 MHz, CDCl₃) δ7.64 (1H, d, J 1.8 Hz), 7.56 (2H, d, J 8.5 Hz), 7.46 (2H, d, J 8.5 Hz), 7.39 (2H, d, J 8.7 Hz), 7.20 (1H, dd, J 8.7, 1.8 Hz), 3.57 (3H, s), 2.98 (2H, t, J 7.1 Hz), and 2.66 (2H, t, J 7.1 Hz),.

### DESCRIPTION 51

### Ethyl 5-Chloro-2-(4-Chlorophenyl)-1-Methyl-1H-Indole-3-Propanimidate Hydrochloride

A solution of 5-chloro-2-(4-chlorophenyl)-1-methyl-1H-indole-3-propanenitrile (Description 50, 0.89 g, 2.7 mmol) in ethanol (30 mL) was added to cooled (−5° C.) ethanolic hydrogen chloride (saturated, 20 mL). The mixture was resaturated with bubbling hydrogen chloride, sealed and stirred at room temperature for 18 h. The solvent was evaporated under reduced pressure and the residue was triturated with ether. The solid was collected and dried in vacuo to give the title compound as a sandy solid (1.1 g, 100%).¹H NMR (360 MHz, CD₃OD) δ7.60 (3H, m), 7.45 (2H, d, J 8.4 Hz), 7.42 (1H, d, J 8.7 Hz), 7.21 (1H, dd, J 8.7, 1.9 Hz), 4.13 (2H, q, J 7.0 Hz), 3.58 (3H, s), 3.16 (2H, t, J 7.2 Hz), 2.80 (2H, t, J 7.2 Hz), and 1.28 (3H, t, J 7.0 Hz). m/z (ES⁺) 375, 377 (M+1).

### DESCRIPTION 52

### 5-Chloro-2-(4-Chlorophenyl)-1-Methyl-1H-Indole

Sodium hydride (60% suspension in mineral oil, 575 mg, 14.3 mmol) was added to a stirred, cooled (0° C.) solution of 5-chloro-2-(4-chlorophenyl)-1H-indole (Description 16, 2.5 g, 9.6 mmol) in dimethylformamide (20 mL) and the mixture was stirred at 0° C. for 5 min. Iodomethane (0.89 mL, 2.0 g, 14.3 mmol) was added and the mixture was stirred at room temperature for 1 h. Water (250 mL) and saturated aqueous ammonium chloride (50 mL) were added and the mixture was extracted with ethyl acetate (2×200 mL). The combined organic fractions were washed with water (2×200 mL), dried (Na₂SO₄) and the solvent was evaporated under reduced pressure. The residue was triturated with EtOAc/hexane (50:50, 20 mL) and the solid was collected and dried in vacuo to give the title compound (1.49 g, 57%). ¹H NMR (360 MHz, CDCl₃) δ7.58 (1H, d, J 1.7 Hz), 7.43 (4H, m), 7.26 (1H, d, J 8.7 Hz), 7.19 (1H, dd, J 8.7, 1.7 Hz), 6.48 (1H, s), and 3.71 (3H, s).

### DESCRIPTION 53

### (RS)-5-Chloro-α-(Chloromethyl)-2-(4-Chlorophenyl)-1H-Indole-3-Ethanol

Tin (IV) chloride (1M in dichloromethane, 5.0 mL, 5.0 mmol) was added dropwise over 5 min. to a stirred, cooled (0° C.) solution of 5-chloro-2-(4-chlorophenyl)-1-methyl-1H-indole (Description 52, 1.16 g, 4.2 mmol) and (RS)-(chloromethyl)oxirane (0.39 mL, 0.47 g, 5.0 mmol) in dichloromethane (30 mL) and the mixture was stirred at room temperature for 20 h. Further (RS)-(chloromethyl)oxirane (0.39 mL, 0.47 g, 5.0 mmol) was added and the mixture was stirred at room temperature for 1 h. Further (RS)-(chloromethyl)oxirane (0.39 mL, 0.47 g, 5.0 mmol) was added and the mixture was stirred at room temperature for 1 h. Saturated aqueous sodium carbonate (50 mL) and water (50 mL) were added slowly and the mixture was extracted with ethyl acetate (2×100 mL). The combined organic fractions were dried (MgSO₄) and the solvent was evaporated under reduced pressure. The residue was purified by flash column chromatography on silica gel, eluting with isohexane/EtOAc (90:10), to give the title compound (1.03 g, 66%). ¹H NMR (360 MHz, CDCl₃) δ7.62 (1H, d, J 1.7 Hz), 7.48 (2H, d, J 8.4 Hz), 7.35 (2H, d, J 8.4 Hz), 7.23 (2H, m), 4.04 (1H, m), 3.55 (3H, s), 3.52 (1H, dd, J 11.1, 3.6 Hz), 3.39 (1H, dd, J 11.1, 6.7 Hz), 2.91 (2H, d, J 5.8 Hz), and 2.13 (1H, d, J 4.5 Hz).

### DESCRIPTION 54

### (RS)-5-Chloro-2-(4-Chlorophenyl)-1H-3-(Oxiranylmethyl)Indole

A mixture of (RS)-5-chloro-α-(chloromethyl)-2-(4-chlorophenyl)-1H-indole-3-ethanol (Description 53, 0.93 g, 2.5 mmol) and potassium carbonate (1.0 g, 7.2 mmol) in acetonitrile (20 mL) was stirred at 80 ° C for 20 h. Further potassium carbonate (0.5 g, 3.6 mmol) and acetonitrile (10 mL) were added and the mixture was stirred at 100° C. for 20 h. The mixture was cooled and water (400 mL) was added. The mixture was extracted with ethyl acetate (500 mL, 150 mL). The combined organic fractions were washed with brine (200 mL), dried (Na₂SO₄) and the solvent was evaporated under reduced pressure. The residue was purified by flash column chromatography on silica gel, eluting with isohexane/EtOAc (90:10 increasing to 75:25), to give the title compound (0.42 g, 50%). ¹H NMR (360 MHz, CDCl₃) δ7.63 (1H, d, J 2.0 Hz), 7.48 (2H, d, J 8.5 Hz), 7.36 (2H, d, J 8.5 Hz), 7.22 (2H, m), 3.56 (3H, s), 3.10 (1H, m), 2.89 (2H, d, J 5.0 Hz), 2.72 (1H, t, J 4.7 Hz), and 2.44 (1H, dd, J 4.7, 2.6 Hz).

### DESCRIPTION 55

### (RS)-5-Chloro-2-(4-Chlorophenyl)-3-(3-Chloro-2-Fluoropropyl)-1H-Indole

(N-Ethylethanaminato)trifluorosulfur (93 ,u, 113 mg, 0.7 mmol) was added to a stirred, cooled (−60 ° C.) solution solution of (RS)-5-chloro-α-(chloromethyl)-2-(4-chlorophenyl)-1H-indole-3-ethanol (Description 53, 117 mg, 0.3 mmol) in ethyl acetate (5 mL) and the mixture was stirred at −60 ° C. for 1 h., then at room temperature for 1 h. Saturated aqueous sodium hydrogen carbonate (5 mL) and water (1 mL) were added and the layers were separated. The aqueous layer was extracted with ethyl acetate (10 mL) and the combined organic fractions were dried (Na₂SO₄) and the solvent was evaporated under reduced pressure. The residue was purified by flash column chromatography on silica gel, eluting with isohexane/EtOAc (95:5), to give the title compound (78 mg, 66%). ¹H NMR (360 MHz, CDCl₃) δ7.60 (1H, d, J 1.7 Hz), 7.49 (2H, d, J 8.4 Hz), 7.32 (2H, d, J 8.4 Hz), 7.25 (2H, m), 4.80 (1H, dpent, Jd 47.3, Jp5.7 Hz), 3.55 (3H, s), 3.52 (2H, m), and 3.12-3.03 (2H, m).

### EXAMPLE 1

### 1′-{3-[5-Chloro-2-(4-Chlorophenyl)-1H-Indol-3-yl]-1-Oxopropyl}-6-(Methylsulfonyl)Spiro[2H-1-Benzopyran-2,4′-Piperidin]-4(3H)-One

Diisopropylethylamine (131 mg) and bromoacetonitrile (430 mg) were added to the resin of Description 3 (150 mg) in N-methylpyrrolidinone (1.2 mL) and the mixture was allowed to stand at room temperature for 24 h. The mixture was filtered and the resin was washed with N-methylpyrrolidinone (5 mL) and tetrahydrofuran (5 mL). A solution of 6-(methylsulfonyl)spiro [2H-1-benzopyran-2,4′-piperidin] -4(3H)-one (PCT Int. Appl. WO 94/17045. Chem. Abstr. 1995, 123, 55696, 24.8 mg) in tetrahydrofuran (1.6 mL) was added and the mixture was allowed to stand at room temperature for 24 h. The mixture was filtered, washing with tetrahydrofuran (0.5 mL) and the filtrate was collected. The solvent was evaporated under reduced pressure and the residue was dried in vacuo to give the title compound (21 mg). ¹H NMR (360 MHz, CDCl₃) δ1.46-1.54 (1H, m), 1.58-1.66 (1H, m), 1.82-1.88 (1H, m), 1.98-2.06 (4H, m), 2.58-2.67 (4H, m), 3.01-3.12 (2H, m), 3.18-3.26 (2H, m), 3.62-3.68 (1H, m), 4.22-4.28 (1H, m), 7.16 (1H, dd, J 8.6, 2.0 Hz), 7.28 (1H, d, J 8.6 Hz), 7.44-7.52 (7H, d, J 8.8 Hz), 7.56(1H, d, J 3.0 Hz) and 8.20 (1H, br).

The following compounds were prepared from the resins of Description 1 or Description 3 according to the method of Example 1, substituting a suitable amine for 6-(methylsulfonyl)spiro[2H-1-benzopyran-2,4′-piperidin]-4(3H)-one.

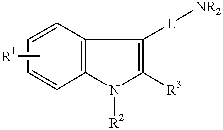

| | | | | | | | | m/z |
|---|---|---|---|---|---|---|---|---|
| | | | | | | | | (ES⁺) |
| | | | | | | | | (M + |
| Ex. | R¹ | R² | R³ | —L— | —NR₂ | Formula | M.W. | 1). |
| 2¹ | 5-Methyl | 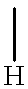 | 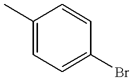 | 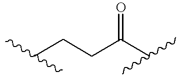 | 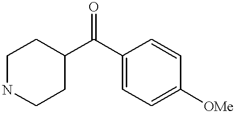 | C31H31Br- N2O3 | 558 560 | 559 561 |
| 3² | 5-Methyl | 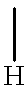 | 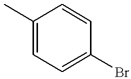 | 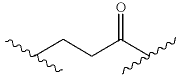 | 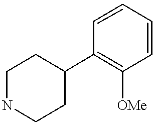 | C3OH31Br- N2O2 | 530 532 | 531 533 |
| 4 | 5-Chloro | 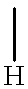 | 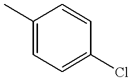 | 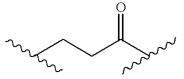 | 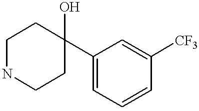 | C29H25Cl2F3- N2O2 | 560 562 | 561 563 |
| 5 | 5-Chloro | 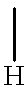 | 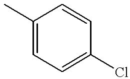 | 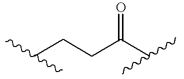 | 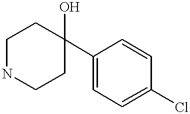 | C28H25Cl3- N2O2 | 526 528 | 527 529 |
| 6 | 5-Chloro | 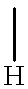 | 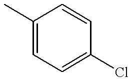 | 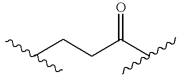 | 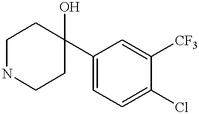 | C29H24Cl3F3- N2O2 | 594 596 | 595 597 |
| 7 | 5-Chloro | 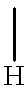 | 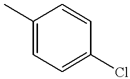 | 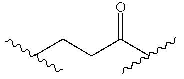 | 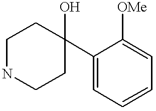 | C29H28Cl2- N2O3 | 522 524 | 523 525 |
| 8 | 5-Chloro | 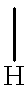 | 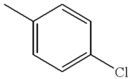 | 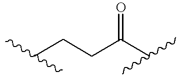 | 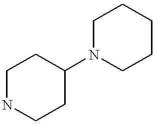 | C27H31Cl2- N3O | 483 485 | 484 486 |
| 9³ | 5-Chloro | 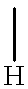 | 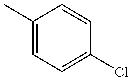 | 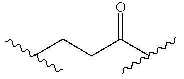 | 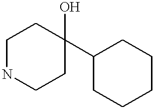 | C28H32Cl2- N2O2 | 498 500 | 499 501 |
| 10 | 5-Chloro | 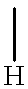 | 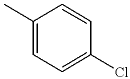 | 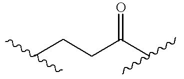 | 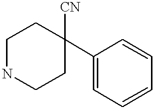 | C29H25Cl2- N3O | 501 503 | 502 504 |
| 11 | 5-Chloro | 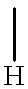 | 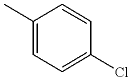 | 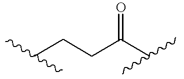 | 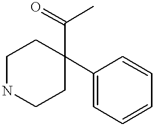 | C30H28Cl2- N2O2 | 518 520 | 519 521 |
| 12⁴ | 5-Chloro | 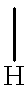 | 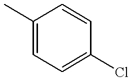 | 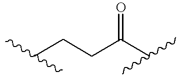 | 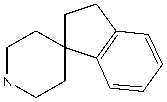 | C30H28Cl2- N2O | 502 504 | 503 505 |
| 13⁵ | 5-Chloro | 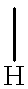 | 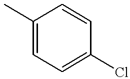 | 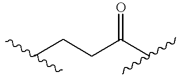 | 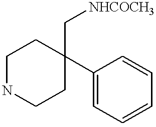 | C31H31Cl2- N3O2 | 547 549 | 548 550 |
| 14⁶ | 5-Chloro | 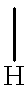 | 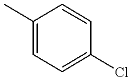 | 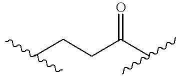 | 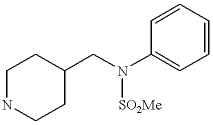 | C30H31Cl2- N3O3S | 583 585 | 584 586 |
| 15⁷ | 5-Chloro | 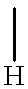 | 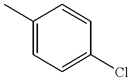 | 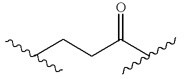 | 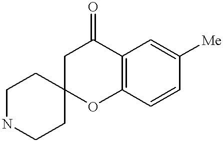 | C31H28Cl2- N2O3 | 546 548 | 547 549 |
| 16⁸ | 5-Chloro | 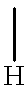 | 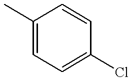 | 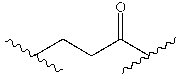 | 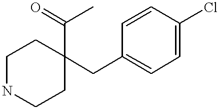 | C31H29Cl3- N2O2 | 566 568 | 567 569 |
| 17⁹ | 5-Chloro | 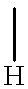 | 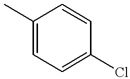 | 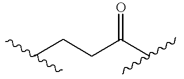 | 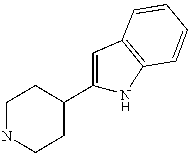 | C30H27Cl2- N3O | 515 517 | 516 518 |
| 18 | 5-Chloro | 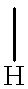 | 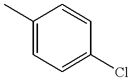 | 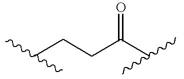 | 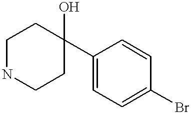 | C28H25BrCl2- N2O2 | 570 572 | 571 573 |
| 19 | 5-Chloro | 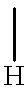 | 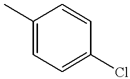 | 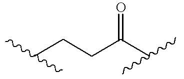 | 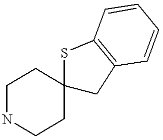 | C29H26Cl2- N2OS | 520 522 | 521 523 |
| 20¹⁰ | 5-Chloro | 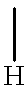 | 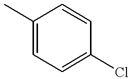 | 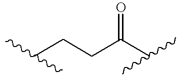 | 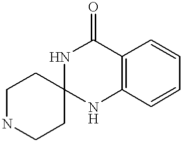 | C29H26Cl2- N4O2 | 532 534 | 533 535 |
| 21 | 5-Chloro | 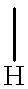 | 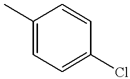 | 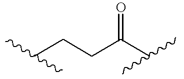 | 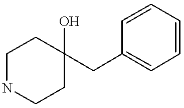 | C29H28Cl2- N2O2 | 506 508 | 507 509 |
| 22 | 5-Chloro | 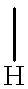 | 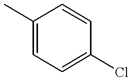 | 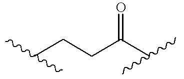 | 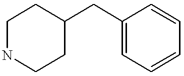 | C29H28Cl2- N2O | 490 492 | 491 493 |
| 23 | 5-Chloro | 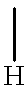 | 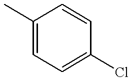 | 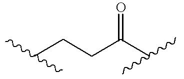 | 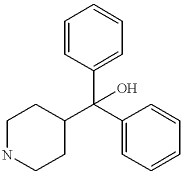 | C35H32Cl2- N2O2 | 582 584 | 583 585 |
| 24¹¹ | 5-Chloro | 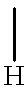 | 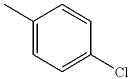 | 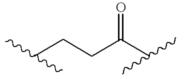 | 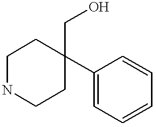 | C29H28Cl2- N2O2 | 506 508 | 507 509 |
| 25 | 5-Chloro | 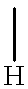 | 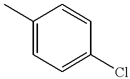 | 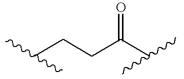 | 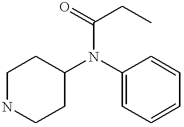 | C31H31Cl2- N3O2 | 547 549 | 548 550 |

¹(4-Methoxyphenyl)-4-piperidinylmethanone hydrochloride; J. Med. Chem. 1970, 13, 1-6.
²4-(2-Methoxyphenyl)piperidine Hydrochloride; Description 30
³4-Cyclohexyl-4-piperidinol; Description 25
⁴2,3-Dihydrospiro[1H-indene-1,4′-piperidine]; J. Med. Chem. 1992, 35, 2033-2039.
⁵N-(4-Phenylpiperidine-4-methyl)acetamide Hydrochloride; Description 33
⁶N-Phenyl-N-(piperidine-4-methyl) methanesulfonamide Hydrochloride; Description 38
⁷6-Methylspiro[2H-1-benzopyran-2,4′-piperidin]-4(3H)-one hydrochloride; PCT Int. Appl. WO 94/13696. Chem. Abstr. 1995, 122, 213945.
⁸1-[4-[(4-Chlorophenyl)methyl]-4-piperidinyl]ethanone hydrochloride; U.S. Pat. No. 5,475,109. Chem. Abstr. 1996, 124, 232250.
⁹2-(4-Piperidinyl)-1H-Indole; PCT Int. Appl. WO 9747302. Chem. Abstr. 1998, 128, 75295.
¹⁰Spiro[piperidine-4,2′(1′H)-quinazolin]-4′(3′H)-one hydrochloride; GB 2309458. Chem. Abstr. 1998, 128, 13439.
¹¹4-Phenyl-4-piperidinemethanol; PCT Int. Appl. WO 94/10165. Chem. Abstr. 1995, 122, 81123.

### EXAMPLE 26

### 1- [3-(5-Methyl-2-phenyl-1H-Indol-3-yl)-1-Oxopropyl]-4-[3-(Trifluoromethyl)Phenyl]-4-Piperidinol

Triethylamine (0.06 mL, 0.4 mmol) was added to a mixture of 5-methyl-2-phenyl-1H-indole-3-propanoic acid (Description 5, 0.1 g, 0.36 mmol), 4-[3-(trifluoromethyl)phenyl]-4-piperidinol (132 mg, 0.54 mmol) and 1-hydroxybenzotriazole (48 mg, 0.4 mmol) in tetrahydrofuran (1 mL) and the mixture was stirred at room temperature for 10 min. 1-(3-Dimethylaminopropyl)-3-ethyl carbodiimide hydrochloride (76 mg, 0.4 mmol) was added and the mixture was stirred at room temperature for 21 h. The mixture was poured into water and extracted with ethyl acetate. Using a Bond Elut™ cartridge to separate the layers the solution was washed with hydrochloric acid (1M), and aqueous sodium hydroxide (2M). The organic fraction was evaporated under reduced pressure to a small volume and filtered through a plug of silica on a Bond Elut™ cartridge, eluting with hexane/EtOAc (85:15 increasing to 70:30), to give the title compound as a colorless solid (100 mg, 55%). ¹H NMR (360 MHz, CDCl₃) δ7.96 (1H, br s), 7.64-7.22 (10H, m), 7.08-7.02 (1H, m), 4.58-4.48 (1H, m), 3.62-3.52 (1H, m), 3.40-3.28 (4H, m), 3.04-2.92 (1H, m), 2.83-2.63 (1H, m), 2.47 (3H, m), 1.76-1.62 (2H, m), and 1.47-1.38 (2H, m). m/z (ES⁺) 507 (M+1).

The following compounds were prepared from 5-methyl-2-phenyl-1H-indole-3-propanoic acid (Description 5) or 5-chloro-2-(4-chlorophenyl)-1-methyl-1H-indole-3-propanoic acid (Description 11) according to the method of Example 26, substituting a suitable amine for 4-[3-(trifluoromethyl)phenyl]-4-piperidinol.

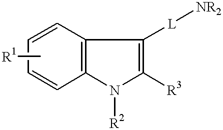

| | | | | | | | | m/z |
|---|---|---|---|---|---|---|---|---|
| | | | | | | | | (ES⁺) |
| | | | | | | | | (M + |
| Ex. | R¹ | R² | R³ | —L— | —NR₂ | Formula | M.W. | 1). |
| 27 | 5-Methyl | 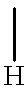 | 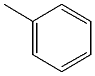 | 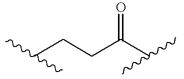 | 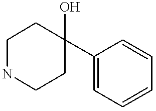 | C29H30N2O2 | 438 | 439 |
| 28¹² | 5-Methyl | 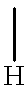 | 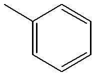 | 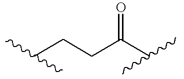 | 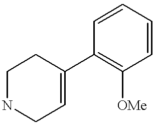 | C30H30N2O2 | 450 | 451 |
| 29¹³ | 5-Methyl | 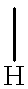 | 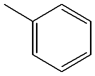 | 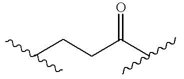 | 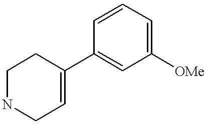 | C30H30N2O2 | 450 | 451 |
| 30¹⁴ | 5-Chloro | 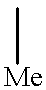 | 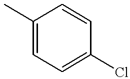 | 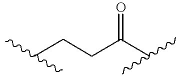 | 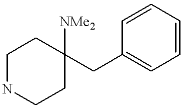 | C32H35Cl2N3O | 547 549 | 548 550 |

¹²1,2,3,6-Tetrahydro-4-(2-methoxyphenyl)-pyridine; PCT Int. Appl. WO 97/28140. Chem. Abstr. 1997, 127, 205594.
¹³1,2,3,6-Tetrahydro-4-(3-methoxyphenyl)-pyridine; PCT Int. Appl. WO 97/28140. Chem. Abstr. 1997, 127, 205594.
¹⁴4-(Dimethylamino)-4-(phenylmethyl)piperidine; Description 28

### EXAMPLE 31

### 1-{3- [5-Chloro-2-(4-Chlorophenyl)-1-Methyl-1H-Indol-3-yl]-1-Oxopropyl}-4-(Phenylmethyl)-4-Piperidinol

1,1-Carbonyl diimidazole (47 mg, 0.29 mmol) was added to a solution of 5-chloro-2-(4-chlorophenyl)-1-methyl-1H-indole-3-propanoic acid (Description 11, 100 mg, 0.29 mmol) in tetrahydrofuran (4 mL) and the mixture was heated under reflux for 2 h. The mixture was cooled and 4-(phenylmethyl)-4-piperidinol (52 mg, 0.27 mmol) was added. The mixture was stirred at room temperature overnight and the solvent was evaporated under reduced pressure. Water (4 mL) was added and the mixture was stirred at 80° C. for 2 h. The mixture was cooled, the water was decanted and the residue was dissolved in dichloromethane. Using a Bond Elut™ cartridge to separate the layers the solution was washed with hydrochloric acid (1M), and aqueous sodium hydroxide (2M). The organic fraction was evaporated under reduced pressure to a small volume and filtered through a plug of silica on a Bond Elut™ cartridge, eluting with hexane/EtOAc (85:15 increasing to 70:30), to give the title compound as a colorless solid (101 mg, 68%). ¹H NMR (360 MHz, CDCl₃) δ1.12-1.18 (2H, m), 1.29 (1H, d, J 13.7 Hz), 1.38-1.42 (2H, m), 1.52 (1H, br), 2.39-2.45 (2H, m), 2.59 (2H, s), 2.75-2.85 (1H, m), 2.89-2.95 (1H, m), 3.11 (1H, dt, J 13.0, 3.3 Hz), 3.29 (1H, m), 3.47 (3H, s), 4.29 (1H, m), 7.07-7.27 (9H, m), 7.40(2H, d, J 6.5 Hz), and 7.53 (1H, s). m/z (ES⁺) 521, 523 (M+1).

The following compounds were prepared from 5-chloro-2-(4-chlorophenyl)-1-methyl-1H-indole-3-propanoic acid (Description 11) or (RS)-α-methyl-[5-chloro-2-(4-chlorophenyl)-1-methyl-1H-indole]-3-propanoic acid (Description 11) according to the method of Example 31, substituting a suitable amine for 4-(phenylmethyl)-4-piperidinol.

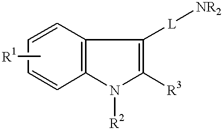

| | | | | | | | | m/z |
|---|---|---|---|---|---|---|---|---|
| | | | | | | | | (ES⁺) |
| | | | | | | | | (M + |
| Ex. | R¹ | R² | R³ | —L— | —NR₂ | Formula | M.W. | 1). |
| 32¹⁵ | 5-Chloro | 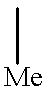 | 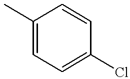 | 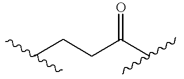 | 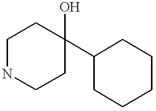 | C29H34Cl2N2O2 | 512 514 | 513 515 |
| 33 | 5-Chloro | 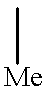 | 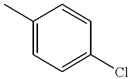 | 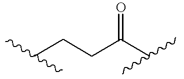 | 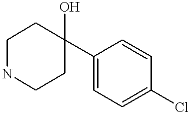 | C29H27Cl3N2O2 | 540 542 | 541 543 |
| 34 | 5-Chloro | 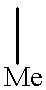 | 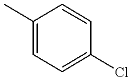 | 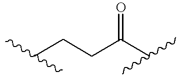 | 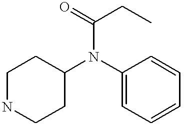 | C32H33Cl2N3O2 | 561 563 | 562 564 |
| 35¹⁶ | 5-Chloro | 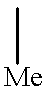 | 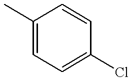 | 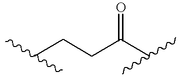 | 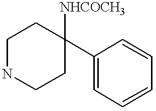 | C31H31Cl2N3O2 | 547 549 | 548 550 |
| 36 | 5-Chloro | 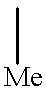 | 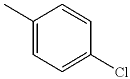 | 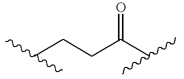 | 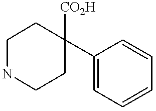 | C30H28Cl2N2O3 | 534 536 | 535 537 |
| 37 | 5-Chloro | 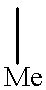 | 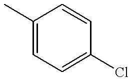 | 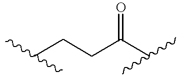 | 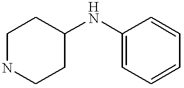 | C29H29Cl2N3O | 505 507 | 506 508 |
| 38 | 5-Chloro | 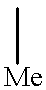 | 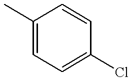 | 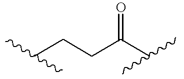 | 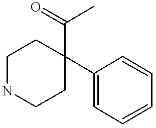 | C31H30Cl2N2O2 | 532 534 | 533 535 |
| 39¹⁷ | 5-Chloro | 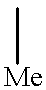 | 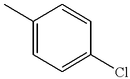 | 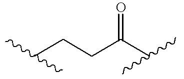 | 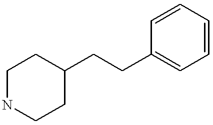 | C31H32Cl2N2O | 518 520 | 519 521 |
| 40¹⁸ | 5-Chloro | 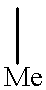 | 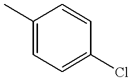 | 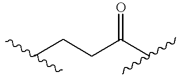 | 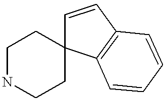 | C31H28Cl2N2O | 514 516 | 515 517 |
| 41¹⁹ | 5-Chloro | 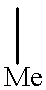 | 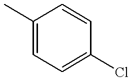 | 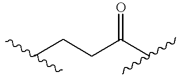 | 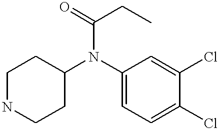 | C32H31Cl4N3O2 | 629 631 | 630 632 |
| 42 | 5-Chloro | 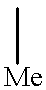 | 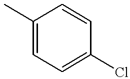 | 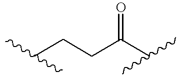 | 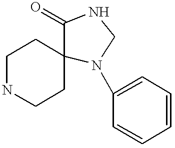 | C31H30Cl2N4O2 | 560 562 | 561 563 |
| 43 | 5-Chloro | 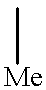 | 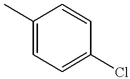 | 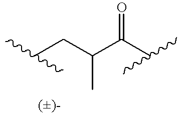 | 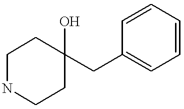 | C31H32Cl2N2O2 | 534 536 | 535 537 |

¹⁵4-Cyclohexyl-4-piperidinol; Description 25.
¹⁶N-(4-Phenyl-4-piperidinyl)acetamide; Eur. Pat. Appl., EP 0474561. Chem. Abstr. 1992, 117, 26590.
¹⁷4-(2-Phenylethyl)piperidine; PCT Int. Appl. WO 94/21627. Chem. Abstr. 1995, 122, 9864.
¹⁸Spiro[1H-indene-1,4′-piperidine]hydrochloride; J. Med. Chem.; 1992, 35, 2033-2039.
¹⁹N-(3,4-dichlorophenyl)-N-4-piperidinylpropanamide; Eur. J. Med. Chem. 1997, 32, 843-868.

### EXAMPLE 44

### 1-{3-[2-(4-Chlorophenyl)-5-Ethenyl-1-Methyl-1H-Indol-3-yl]-1-Oxopropyl}-4-(Phenylmethyl)-4-Piperidinol

Lithium hydroxide monohydrate (15 mg, 0.37 mmol) was added to a solution of methyl 2-(4-chlorophenyl)- 5-ethenyl-1-methyl-1H-indole-3-propanoate (Description 14, 108 mg, 0.31 mmol) in methanol-water (3:1, 4 mL) and the mixture was stirred at room temperature overnight. Tetrahydrofuran (1 mL) and further portions of lithium hydroxide monohydrate were added at intervals until TLC showed no starting material (5 equivalents added altogether). The solvent was evaporated under reduced pressure and the residue was dissolved in tetrahydrofuran (15 mL). 1-Hydroxybenzotriazole (737 mg, 2.48 mmol), triethylamine (344 μl, 2.48 mmol), 1-(3-dimethylaminopropyl)-3-ethylcarbodiimide hydrochloride (475 mg, 2.48 mmol) and 4-(phenylmethyl)-4-piperidinol (474 mg, 2.48 mmol) were added and the mixture was stirred at room temperature overnight. The mixture was poured into water (25 mL) and extracted with ethyl acetate (2×25 mL). The combined organic fractions were washed with aqueous sodium carbonate (10%, 25 mL), hydrochloric acid (1M, 25 mL) and brine (25 mL), dried (MgSO₄) and the solvent was evaporated under reduced pressure . The residue was purified by flash column chromatography on silica gel, eluting with ethyl acetate-hexane (3:2), to give the title compound (44 mg, 28%). ¹H NMR (360 MHz, CDCl₃) δ7.63 (1H, s), 7.48-7.12 (11H, m), 6.87 (1H, dd, J 17.6, 10.9 Hz), 5.74 (1H, dd, J 17.6, 0.7 Hz), 5.18 (1H, dd, J 10.9, 0.7 Hz), 4.35 (1H, br d, J 14.2 Hz), 3.55 (3H, s), 3.34 (1H, br d, J 12.3 Hz), 3.16 (1H, dt, Jₜ 12.8, J_{d} 2.8 Hz), 3.07-3.01 (2H, m), 2.89-2.81 (1H, m), 2.63 (2H, s), 2.54-2.49 (2H, m), 1.45-1.41 (2H, m), 1.32 (1H, br d, J 13.3 Hz), and 1.14 (1H, dt, Jₜ 12.8, J_{d} 4.6 Hz).

### EXAMPLE 45

### Methyl 2-(4-Chlorophenyl)-3-(3-[4-Hydroxy-4-(Phenylmethyl)Piperidin-1-yl]-3-Oxopropyl)-1-Methyl-1H-Indol-5-Carboxylate

Prepared from methyl 2-(4-chlorophenyl)-1-methyl-5-(methoxycarbonyl)-1H-indole-3-propanoate (Description 12) according to the method of Example 44. ¹H NMR (360 MHz, CDCl₃) δ8.39 (1H, d, J 1.5 Hz), 7.97 (1H, dd, J 5.1, 1.5 Hz), 7.50-7.14 (1OH, m), 4.35 (1H, br d, J 12.8 Hz), 3.95 (3H, s), 3.58 (3H, s), 3.39 (1H, br d, J 13.0 Hz), 3.23-3.17 (1H, m), 3.08-3.04 (2H, m), 2.92-2.84 (1H, m), 2.69 (2H, s), 2.57-2.53 (2H, m), and 1.52-1.33 (4H, m).

### EXAMPLE 46

### (E)-1-{3- [5-Chloro-2-(4-Chlorophenyl)-1-Methyl-1H-Indol-3-yl]-1-Oxo-2-Propenyl}-4-(Phenylmethyl)-4-Piperidinol

Prepared from (E)-ethyl [5-chloro-2-(4-chlorophenyl)-1-methyl-1H-indol-3-yl]-2-propenoate (Description 19) according to the method of Example 44. ¹H NMR (360 MHz, CDCl₃) δ7.86 (1H, d, J 1.6 Hz), 7.63 (1H, d, J 15.4 Hz), 7.52-7.50 (2H, m), 7.32-7.19 (9H, m), 6.79 (1H, d, J 15.4 Hz), 4.46 (1H, m), 3.86 (1H, m), 3.61 (3H, s), 3.48 (1H, m), 3.06 (1H, m), 2.78 (2H, s), and 1.67-1.56 (4H, m).

### EXAMPLE 47

### (Z)-1-{3-[5-Chloro-2-(4-Chlorophenyl)-1-Methyl-1H-Indol-3-yl]-1-Oxo-2-Propenyl}-4-(Phenylmethyl)-4-Piperidinol

Prepared from (Z)-methyl [5-chloro-2-(4-chlorophenyl)-1-methyl-1H-indol-3-yl]-2-propenoate (Description 20) according to the method of Example 44. ¹H NMR (360 MHz, CDCl₃) δ7.56 (1H, d, J 1.5 Hz), 7.49 (2H, d, J 8.2 Hz), 7.34-7.21 (7H, m), 7.12 (2H, d, J 8.2 Hz), 6.52 (1H, d, J 12.4 Hz), 5.97 (1H, d, J 12.4 Hz), 4.40 (1H, m), 3.73 (1H, m), 3.62 (3H, s), 3.17 (1H, m), 2.89 (1H, m), 2.63 (2H, s), and 1.57-1.24 (5H, m). m/z (ES⁺) 519, 521 (M+1).

### EXAMPLE 48

### 1-{3- [5-Chloro-2-(4-Chlorophenyl)-1-Methyl-1H-Indol-3-yl]-1-Oxo-2-Propenyl}-4-(Phenylmethyl)-4-Piperidinol

Prepared from methyl [5-chloro-2-(4-chlorophenyl)-1-methyl-1H-indol-3-yl]-2-propynoate (Description 22) according to the method of Example 44. ¹H NMR (360 MHz, CDCl₃) δ7.77 (1H, m), 7.51 (4H, s), 7.36-19 (7H, m), 4.38 (1H, br d, J 12.0 Hz), 3.98 (iH, br d, J 12.0 Hz), 3.71 (3H, s), 3.40-3.30 (1H, m), 3.08-2.99 (1H, m), 2.77 (2H, s), and 1.62-1.46 (4H, m).

### EXAMPLE 49

### 1-{3- [5-Bromo-2-(4-Chlorophenyl)-1-Methyl-1H-Indol-3-yl]-1-Oxopropyl}-4-(Phenylmethvl)-4-Piperidinol

Prepared from methyl 5-bromo-2-(4-chlorophenyl)-1-methyl-1H-indole-3-propanoate (Description 13) according to the method of Example 44. ¹H NMR (360 MHz, CDCl₃) δ7.75 (1H, d, J 1.9 Hz), 7.49-7.47 (2H, m), 7.35-7.14 (9H, m), 4.36 (1H, br d, J 12.6 Hz), 3.54 (3H, s), 3.37 (1H, br d, J 13.7 Hz), 3.18 (1H, m), 3.02-2.96 (2H, m), 2.90-2.84 (1H, m), 2.66 (2H, s), 2.52-2.47 (2H, m), 1.49-1.46 (2H, m), 1.37 (1H, br d, J 13.4 Hz), and 1.26-1.21 (1H, m).

### EXAMPLE 50

### 1-{3-[5-Acetyl-2-(4-Chlorophenyl)-1-Methyl-1H-Indol-3-yl]-1-Oxopropyl}-4-(Phenylmethyl)-4-Piperidinol

A mixture of palladium chloride (2 mg, 0.012 mmol) and copper (I) chloride (12 mg, 0.12 mmol) in dimethylformamide-water (7:1, 11 mL) was stirred under oxygen at room temperature for 1 h. A solution of 1-{3- [2-(4-chlorophenyl)-5-ethenyl-1-methyl-1H-indol-3-yl] -1-oxopropyl}-4-(phenylmethyl)-4-piperidinol (Example 44, 60 mg, 0.12 mmol) in dimethylformamide-water (7:1, 5 mL) was added dropwise over 5 min. and the mixture was stirred under oxygen at room temperature for 24 h. The mixture was poured into hydrochloric acid (2M, 100 mL) and extracted with ether (5×30 mL). The combined organic fractions were washed with aqueous sodium hydrogen carbonate (saturated, 30 mL) and brine (30 mL), dried (MgSO₄) and the solvent was evaporated under reduced pressure. The residue was purified by flash column chromatography on silica gel, eluting with ethyl acetate: hexane (1:1), to give the title compound. ¹H NMR (360 MHz, CDCl₃) δ8.32 (1H, d, J 1.6 Hz), 7.94 (1H, dd, J 8.6, 1.6 Hz), 7.51-7.47 (2H, m), 7.36-7.24 (7H, m), 7.14 (1H, d, J 8.6 Hz), 4.34 (1H, br d, J 12.9 Hz), 3.59 (3H, s), 3.37 (1H, br d, J 13.4 Hz), 3.18 (1H, dt, Jt 12.5, Jd 2.9 Hz), 3.12-3.05 (2H, m), 2.90-2.82 (1H, m), 2.69 (3H, s), 2.66 (2H, s), 2.55-2.50 (2H, m), and 1.45-1.19 (4H, m). m/z (ES⁺) 529, 531 (M+1).

### EXAMPLE 51

### 1-{3- [2-(4-Chlorophenyl)-1-Methyl-5-(3-Pyridinyl)-1H-Indol-3-yl]-1-Oxopropyl}-4-(Phenylmethyl)-4-Piperidinol

Prepared from 1-{3-[5-bromo-2-(4-chlorophenyl)-1-methyl-1H-indol-3-yl]-1-oxopropyl}-4-(phenylmethyl)-4-piperidinol (Example 49) and 3-(tributylstannyl)pyridine, according to the method of Description 14. ¹H NMR (360 MHz, CDCl₃) δ8.93 (1H, d, J 1.7 Hz), 8.56 (1H, m), 8.00 (1H, m), 7.85 (11, d, J 1.7 Hz), 7.53-7.25 (10H, m), 7.12 (2H, dd, J 8.1, 1.6 Hz), 4.34 (1H, br d, J 13.0 Hz), 3.61 (3H, s), 3.36 (1H, br d, J 13.2 Hz), 3.20-3.07 (3H, m), 2.89-2.81 (1H, m), 2.63 (2H, s), 2.57-2.52 (2H, m), and 1.42-1.14 (4H, m). m/z (ES⁺) 564, 566 (M+1).

### EXAMPLE 52

### 1-{3- [2-(4-Chlorophenyl)-5-(2-Furanyl)-1-Methyl-1H-Indol-3-yl]-1-Oxopropyl}-4-(Phenylmethyl)-4-Piperidinol

2-Furanylboronic acid (40 mg, 0.36 mmol) and aqueous sodium carbonate (2M, 1 mL) were added to a solution of 1-{3-[5-bromo-2-(4-chlorophenyl)-1-methyl-1H-indol-3-yl]-1-oxopropyl}-4-(phenylmethyl)-4-piperidinol (Example 49, 100 mg, 0.18 mmol) in 1,2-dimethoxyethane (10 mL) and the mixture was degassed. Tris(dibenzylideneacetone) dipalladium(0) (66 mg) and tris(2-furanyl)phosphine (2 mg) were added and the mixture was degassed, then heated to 75° C. overnight. The mixture was cooled and poured into aqueous sodium hydrogen carbonate (saturated, 50 mL) and extracted with ethyl acetate (4×25 mL). The combined organic fractions were washed with brine, dried (MgSO₄) and the solvent was evaporated under reduced pressure . The residue was purified by medium pressure liquid chromatography on silica gel, eluting with ethyl acetate: isohexane (1:1), to give the title compound (30 mg, 30%). ¹H NMR (360 MHz, CDCl₃) δ7.95 (1H, d, J 1.7 Hz), 7.61 (1H, dd, J 1.6, 8.3 Hz), 7.49-7.47 (3H, m), 7.34-7.26 (6H, m), 7.12 (2H, dd, J 1.6, 8.3 Hz), 6.63 (1H, d, J 3.2 Hz), 4.34 (1H, br d, J 12.6 Hz), 3.57 (3H, s), 3.37 (1H, br d, J 10.5 Hz), 3.18 (1H, dt, Jₜ 11.3, J_{d} 1.5 Hz), 3.10-3.04 (2H, m), 2.90-2.82 (1H, m), 2.63 (2H, s), 2.58-2.52 (2H, m), 1.47-1.42 (2H, m), 1.34 (1H, br d, J 13.8 Hz), and 1.22-1.16 (1H, m). m/z (ES⁺) 553, 555 (M+1).

### EXAMPLE 53

### N-Methyl-N-Phenyl-1-{3-[5-Chloro-2-(4-Chlorophenyl)-1-Methyl-1H-Indol-3-yl]-1-Oxopropyl}-4-Piperidinamine

Aqueous formaldehyde (37%, 80 μl) was added to a solution of N-Phenyl-1-{3- [5-chloro-2-(4-chlorophenyl)-1-methyl-1H-indol-3-yl]-1-oxopropyl}-4-piperidinamine (Example 37, 100 mg, 0.2 mmol) in acetonitrile and the mixture was stirred at room temperature for 15 min. Sodium cyanoborohydride (20 mg) was added and the pH was adjusted to 7.0 by the addition of acetic acid. The mixture was stirred at room temperature for 45 min., maintaining the pH at 7.0 by the addition of further acetic acid. The solvent was evaporated under reduced pressure and aqueous sodium hydrogen carbonate (saturated, 10 mL) and ethyl acetate (25 mL) were added. The layers were separated and the organic fraction was dried (MgSO₄) and the solvent was evaporated under reduced pressure. The residue was purified by flash column chromatography on silica gel, eluting with hexane/EtOAc (80:20 increasing to 50:50) to give the title compound as a colorless foam (75 mg, 72%). ¹H NMR (360 MHz, CDCl₃) δ1.22-1.31 (1H, m), 1.46-1.76 (4H, m), 2.47-2.56 (3H, m), 2.66(3H, s), 2.87-2.94 (1H, m), 2.98-3.04 (2H, m), 3.54 (3H, s), 4.70-4.77 (1H, m), 6.71-6.79 (3H, m), 7.18-7.25 (4H, s), 7.31 (2H, d, J 6.5 Hz), 7.47 (2H, d, J 6.5 Hz), and 7.59 (1H, d, J 1.2 Hz). m/z (ES⁺) 520, 522 (M+1).

### EXAMPLE 54

### 1-{3-[5-Chloro-2-(4-Chlorophenyl)-1-Methyl-1H-Indol-3-yl]Propyl}-4-(Phenylmethyl)-4-Piperidinol Hydrochloride

Sodium triacetoxyborohydride (292 mg, 1.35 mmol) was added to a mixture of 5-chloro-2-(4-chlorophenyl)-1-methyl-1H-indole-3-propanal (Description 23, 91 mg, 0.27 mmol), 4-(phenylmethyl)-4-piperidinol (54 mg, 0.27 mmol) and glacial acetic acid (77 μL, 1.35 mmol) in 1,2-dichloroethane (5 mL) and the mixture was stirred at room temperature for 2 h. Aqueous sodium hydrogen carbonate (saturated, 40 mL) was added and the mixture was extracted with ethyl acetate (2×40 mL). The combined organic fractions were washed with brine, dried (MgSO₄) and the solvent was evaporated under reduced pressure. The residue was purified by flash column chromatography on silica gel, eluting with CH₂Cl₂/MeOH/NH₃(Aq.) (120:8:1). The residue was dissolved in methanol, ethereal hydrogen chloride solution (1M) was added and the solvent was evaporated under reduced pressure. The residue was crystallized from ethyl acetate to give the title compound (46 mg, 31%). ¹H NMR (360 MHz, DMSO-d₆) δ1.51-1.55 (2H, m), 1.65-1.80 (2H, m) 1.82-1.96 (2H, m), 2.57-2.68 (2H, m), 2.70 (2H, s), 2.90-3.05 (4H, m), 3.54 (3H, s), 4.79 (1H, s), 7.18-7.28 (6H, m), 7.50 (3H, t, J 8.3 Hz), 7.60 (2H, d, J 8.5 Hz), 7.07 (1H, m), and 9.44 (1H, br s). m/z (ES⁺) 507, 509 (M+1).

### EXAMPLE 55

### 1-{3-[5-Chloro-2-(4-Chlorophenyl)-1H-Indol-3-yl]Propyl}-4-(Phenylmethyl)-4-Piperidinol Hydrochloride

Borane tetrahydrofuran complex (1.0M in tetrahydrofuran, 6 mL, 6 mmol) was added to a solution of 1-{3-[5-chloro-2-(4-chlorophenyl)-1H-indol-3-yl]-1-oxopropyl}-4-(phenylmethyl)-4-piperidinol (Example 21, 785 mg, 1.5 mmol) in tetrahydrofuran (15 mL) and the mixture was heated under reflux for 1 h. The mixture was cooled in ice and methanol (5 mL) was added slowly. The solvent was evaporated under reduced pressure, potassium carbonate (0.83 g, 6 mmol) and methanol (20 mL) were added and the mixture was heated under reflux for 12 h. The mixture was cooled, poured into water (50 mL) and extracted with dichloromethane (3×50 mL). The combined organic fractions were dried (MgSO₄) and the solvent was evaporated under reduced pressure. The residue was purified by flash column chromatography on silica gel, eluting with CH₂Cl₂/MeOH/NH₃(Aq.) (98:2:0.2 increasing to 95:5:0.5) to give a tan foam (574 mg). A sample (99 mg, 0.2 mmol) was dissolved in ethanol (2 mL) and ethereal hydrogen chloride (1.0M, 0.3 mL, 0.3 mmol) was added. The solvent was evaporated under reduced pressure and the residue was triturated with ether/2-propanol (20:1, 20 mL). The solid was collected and dried in vacuo to give the title compound as an off-white solid (91 mg, 64%). m.p. 147-150° C. ¹H NMR (400 MHz, CD₃OD) δ7.60 (1H, d, J 2.0 Hz), 7.58 (2H, d, J 8.5 Hz), 7.50 (2H, d, J 8.5 Hz), 7.33 (1H, d, J 8.6 Hz), 7.25 (5H, m), 7.10 (1H, dd, J 8.6, 2.0 Hz), 4.85 (3H, br s), 3.24 (2H, m), 3.12 (2H, m), 3.04 (2H, m), 2.94 (2H, t, J 7.6 Hz), 2.79 (2H, s), 2.04 (2H, m), 1.82 (2H, m), and 1.68 (2H, m). m/z (ES⁺) 493, 495 (M+1).

### EXAMPLE 56

### 1-{3- [1-Acetyl-5-Chloro-2-(4-Chlorophenyl)-1H-Indol-3-yl]Propyl}-4-(Phenylmethyl)-4-Piperidinol

Sodium hydride (60% in mineral oil, 44 mg, 1.1 mmol) was added to a stirred, cooled (0° C.) solution of 1-{3-[5-chloro-2-(4-chlorophenyl)-1H-indol-3-yl]propyl}-4-(phenylmethyl)-4-piperidinol (Example 55, 364 mg, 0.74 mmol) in tetrahydrofuran (5 mL) and the mixture was stirred at 0° C. for 1 h. Acetic anhydride (104 μL, 113 mg, 1.1 mmol) was added and the mixture was stirred at room temperature for 2 h. Aqueous sodium hydrogen carbonate (saturated, 30 mL) and water (10 mL) were added and the mixture was extracted with dichloromethane (3×30 mL). The combined organic fractions were dried (MgSO₄) and the solvent was evaporated under reduced pressure. The residue was purified by flash column chromatography on silica gel, eluting with CH₂Cl₂/MeOH/NH₃(Aq.) (98:2:0.2) to give the title compound as an off-white foam (278 mg, 70%). ¹H NMR (400 MHz, CCl₃) δ8.33 (1H, d, J 8.9 Hz), 7.56 (1H, d, J 2.0 Hz), 7.47 (2H, d, J 8.4 Hz), 7.41-7.23 (6H, m), 7.20 (2H, d, J 8.4 Hz), 2.75 (2H, s), 2.51 (4H, m), 2.26 (2H, m), 2.18 (2H, m), 1.97 (3H, s), 1.69 (4H, m), 1.51 (1H, br s), and 1.49 (2H, m). m/z (ES⁺) 535, 537 (M+1).

### EXAMPLE 57

### 1-{3- [5-Chloro-2-(4-Fluorophenyl)-1-Methyl-1H-Indol-3-yl]-1-Oxopropyl}-4-(Phenylmethyl)-4-Piperidinol

Prepared from methyl 5-chloro-2-(4-fluorophenyl)-1-methyl-1H-indole-3-propanoate (Description 42) according to the method of Example 44. ¹H NMR (360 MHz, CDCl₃) δ7.59 (1H, d, J 1.7 Hz), 7.37-7.13 (11H, m), 4.38-4.34 (1H, m), 3.54 (3H, s), 3.37 (1H, m), 3.21-3.15 (1H, m), 3.02-2.96 (2H, m), 2.86 (1H, m), 2.66 (2H, s), 2.52-2.47 (2H, m), 1.57 (1H, br s), 1.47 (2H, m), 1.36 (1H, m), and 1.28-1.16 (1H, m). m/z (ES⁺) 505, 507 (M+1).

### EXAMPLE 58

### 1-{3- [5-Chloro-1-Methyl-2-(2-Pyridinyl)-1H-Indol-3-yl]-1-Oxopropyl}-4-(Phenylmethyl)-4-Piperidinol

Prepared from methyl 5-chloro-1-methyl-2-(2-pyridinyl)-1H-indole-3-propanoate (Description 43) according to the method of Example 44. ¹H NMR (360 MHz, CDCl₃) δ8.77 (1H, d, J 4.0 Hz), 7.83 (1H, t, J 7.8 Hz), 7.62 (1H, d, J 1.8 Hz), 7.53 (1H, d, J 7.8 Hz), 7.35-7.13 (8H, m), 4.35 (1H, m), 3.72 (3H, s), 3.43 (1H, m), 3.12 (3H, m), 2.88 (1H, m), 2.65 (2H, s), 2.60 (2H, m), 1.58 (1H, br s), and 1.49-1.17 (4H, m). miz (ES⁺) 488, 490 (M+1).

### EXAMPLE 59

### 1-{3- [5-Chloro-1-Methyl-2-(3-Pyridinyl)-1H-Indol-3-yl]-1-Oxopropyl}-4-(Phenylmethyl)-4-Piperidinol Hydrochloride

Prepared from methyl 5-chloro-1-methyl-2-(3-pyridinyl)-1H-indole-3-propanoate (Description 44) according to the method of Example 44. ¹H NMR (360 MHz, CDCl₃) δ9.02 (1H, s), 8.91 (1H, d, J 4.8 Hz), 8.44 (1H, d, J 8.0 Hz), 8.00 (1H, dd, J 8.0, 4.8 Hz), 7.71 (1H, d, J 1.9 Hz), 7.58 (1H, d, J 8.7 Hz), 7.28-7.15 (6H, m), 4.60 (2H, br s), 3.60 (3H, s), 3.41 (1H, m), 3.08 (1H, m), 2.84-2.47 (8H, m), and 1.29-1.08 (4H, m). m/z (ES⁺) 488, 490 (M+1).

### EXAMPLE 60

### 1-{3- [5-Chloro-1-Methyl-2-(6-Methyl-3-Pyridinyl)-1H-Indol-3-yl]-1-Oxopropyl}-4-(Phenylmethyl)-4-Piperidinol

Prepared from methyl 5-chloro-1-methyl-2-(4-methyl-3-pyridinyl)-1H-indole-3-propanoate (Description 48) according to the method of Example 44. ¹H NMR (360 MHz, CDCl₃) δ8.51 (1H, s), 7.61 (2H, m), 7.34-7.14 (8H, m), 4.36 (1H, m), 3.56 (3H, s), 3.39 (1H, m), 3.19 (1H, m), 2.99 (2H, m), 2.87 (1H, m), 2.66 (2H, s), 2.65 (3H, s), 2.53 (2H, m), and 1.66-1.18 (5H, m). m/z (ES⁺) 502, 504 (M+1).

### EXAMPLE 61

### 1-(3-{5-Chloro-1-Methyl-2-[5-(Trifluoromethyl)-2-Pyridinyl]-1H-Indol-3-yl}- 1-Oxopropyl)-4-(Phenylmethyl)-4-Piperidinol

Prepared from methyl 5-chloro-1-methyl-2-[5-(trifluoromethyl)-2-pyridinyl]-1H-indole-3-propanoate (Description 49) according to the method of Example 44. ¹H NMR (360 MHz, CDCl₃) δ9.03 (1H, d, J 2.2 Hz), 8.07 (1H, dd, J 8.4, 2.2 Hz), 7.72 (1H, d, J 8.4 Hz), 7.35-7.24 (5H, m), 7.14 (2H, d, J 6.7 Hz), 4.36 (1H, m), 3.76 (3H, s), 3.46 (1H, m), 3.25-3.08 (3H, m), 2.88 (1H, m), 2.72-2.52 (2H, m), 2.66 (2H, s), and 1.58-1.17 (5H, m). m/z (ES⁺) 556, 558 (M+1).

### EXAMPLE 62

### 1-{3- [2-(4-Chlorophenyl)-1-Methyl-5-(2-Pyridinyl)-1H-Indol-3-yl]-1-Oxopropyl}-4-(Phenylmethyl)-4-Piperidinol

Prepared from 1-{3-[5-bromo-2-(4-chlorophenyl)-1-methyl-1H-indol-3-yl]-1-oxopropyl}-4-(phenylmethyl)-4-piperidinol (Example 49) and 2-(tributylstannyl)pyridine, according to the method of Description 14. ¹H NMR (360 MHz, CDCl₃) δ8.68 (1H, m), 8.25 (1H, d, J 1.0 Hz), 7.99 (2H, dd, J 8.4, 1.0 Hz), 7.84-7.71 (4H, m), 7.49-7.10 (8H, m), 4.34 (1H, m), 3.60 (3H, s), 3.37 (1H, m), 3.19-3.08 (3H, m), 2.85 (1H, m), 2.63 (2H, s), 2.59-2.54 (2H, m), and 1.42-1.16 (5H, m). m/z (ES⁺) 564, 566 (M+1).

### EXAMPLE 63

### 1-{3- [2-(4-Chlorophenyl)-1-Methyl-5-(4-Pyridinyl)-1H-Indol-3-yl]-1-Oxopropyl}-4-(Phenylmethyl)-4-Piperidinol

Prepared from 1-{3-[5-bromo-2-(4-chlorophenyl)-1-methyl-1H-indol-3-yl]-1-oxopropyl}-4-(phenylmethyl)-4-piperidinol (Example 49) and 4-pyridylboronic acid, according to the method of Example 52. ¹H NMR (360 MHz, CDCl₃) δ8.98-8.82 (4H, br m), 7.96 (1H, d, J 1.5 Hz), 7.77-7.24 (9H, m), 7.11 (2H, d, J 6.4 Hz), 4.35 (1H, m), 3.61 (3H, s), 3.36 (1H, m), 3.11 (3H, m), 2.86 (1H, m), 2.62 (2H, s), 2.53 (2H, m), and 1.48-1.10 (5H, m). m/z (ES⁺) 564, 566 (M+1).

### EXAMPLE 64

### 1-{3- [2-(4-Chlorophenyl)-1-Methyl-5-Morpholino-1H-Indol-3-yl]-1-Oxopropyl}-4-(Phenylmethyl)-4-Piperidinol

A mixture of 1-{3-[5-bromo-2-(4-chlorophenyl)-1-methyl-1H-indol-3-yl]-1-oxopropyl}-4-(phenylmethyl)-4-piperidinol (Example 49, 100 mg, 0.18 mmol), morpholine (18 μl, 18 mg, 0.21 mmol), 2,2′-bis(diphenylphosphino)-1,1′-binaphthyl (0.8 mg, 1.3 μmol) and sodium tert-butoxide (24 mg, 0.25 mmol) in toluene (5 mL) was degassed with bubbling nitrogen and tris(dibenzylideneacetone)dipalladium(0) (0.5 mg, 0.5 μmol) was added. The mixture was degassed with bubbling nitrogen, then heated to 80° C. for 8 h. The mixture was cooled and further 2,2′-bis(diphenylphosphino)-1,1′-binaphthyl (0.8 mg, 1.3 μmol) and tris(dibenzylideneacetone) dipalladium(0) (0.5 mg, 0.5 μmol) were added. The mixture was degassed with bubbling nitrogen, then heated to 100° C. for 18 h. The mixture was cooled and the solvent was evaporated under reduced pressure. Water (25 mL) was added and the mixture was extracted with ethyl acetate (2×25 mL). The combined organic fractions were washed with brine, dried (MgSO₄) and the solvent was evaporated under reduced pressure. The residue was dissolved in methanol (1.5 mL) and poured onto an SCX cartridge (Varian Bond Elut; 10 mL/500 mg). The cartridge was washed with methanol (4×1.5 mL) and eluted with methanolic ammonia (2M, 3×1.5 mL). The solvent was evaporated under reduced pressure and the residue was purified by medium pressure liquid chromatography on silica gel, eluting with CH₂Cl₂/MeOH/NH₃(Aq.) (97:3:0.3), to give the title compound (20 mg, 19%). ¹H NMR (360 MHz, CDCl₃) δ7.46 (2H, d, J 8.4 Hz), 7.35-7.25 (6H, m), 7.12 (3H, m), 7.03 (1H, dd, J 8.8, 2.2 Hz), 4.34 (1H, m), 3.92 (4H, t, J 4.7 Hz), 3.53 (3H, s), 3.33 (1H, m), 3.16 (4H, t, J 4.7 Hz), 3.15 (1H, m), 3.03 (2H, m), 2.84 (1H, m), 2.62 (2H, s), 2.51-2.46 (2H, m), 1.42-1.38 (2H, m), 1.31 (1H, m), 1.20 (1H, br s), and 1.10 (1H, m). m/z (ES⁺) 572, 574 (M+1).

### EXAMPLE 65

### 1-{3- [2-(4-Chlorophenyl)-1-Methyl-5-Piperidino-1H-Indol-3-yl]-1-Oxopropyl}-4-(Phenylmethyl)-4-Piperidinol

Prepared from 1-{3- [5-bromo-2-(4-chlorophenyl)-1-methyl-1H-indol-3-yl]-1-oxopropyl}-4-(phenylmethyl)-4-piperidinol (Example 49) and piperidine, according to the method of Example 64. ¹H NMR (360 MHz, CDCl₃) δ7.47-7.43 (2H, m), 7.34-7.23 (6H, m), 7.17-7.13 (3H, m), 7.07 (1H, dd, J 8.8, 2.6 Hz), 4.34 (1H, m), 3.52 (3H, s), 3.34 (1H, m), 3.19-3.10 (5H, m), 3.05-2.99 (2H, m), 2.85 (1H, m), 2.63 (2H, s), 2.53-2.47 (2H, m), 1.82-1.76 (4H, m), 1.62-1.58 (3H, m), 1.43-1.39 (2H, m), 1.31 (1H, m), and 1.18-1.10 (1H, m). m/z (ES⁺) 570, 572 (M+1).

### EXAMPLE 66

### 1-{3-[2-(4-Chlorophenyl)-1-Methyl-5-Pyrrolidino-1H-Indol-3-yl]-1-Oxopropyl}-4-(Phenylmethyl)-4-Piperidinol

Prepared from 1-{3-[5-bromo-2-(4-chlorophenyl)-1-methyl-1H-indol-3-yl]-1-oxopropyl}-4-(phenylmethyl)-4-piperidinol (Example 49) and pyrrolidine, according to the method of Example 64. ¹H NMR (360 MHz, CDCl₃) δ7.44 (2H, d, J 7.4 Hz), 7.34-7.20 (6H, m), 7.13 (2H, d, J 7.4 Hz), 6.75 (2H, m), 4.36 (1H, m), 3.51 (3H, s), 3.36-3.32 (5H, m), 3.16 (1H, m), 3.02 (2H, m), 2.86 (1H, m), 2.63 (2H, s), 2.52 (2H, m), 2.06-2.03 (4H, m), and 1.60-1.10 (5H, m). m/z (ES⁺) 556, 558 (M+1).

### EXAMPLE 67

### 1-{3- [5-Chloro-2-(4-Chlorophenyl)- 1-Methyl-1H-Indol-3-yl]-1-Iminopropyl}-4-(Phenylmethyl)-4-Piperidinol

4-(Phenylmethyl)-4-piperidinol (38 mg, 0.2 mmol) was added to a solution of ethyl 5-chloro-2-(4-chlorophenyl)-1-methyl-1H-indole-3-propanimidate hydrochloride (Description 51, 100 mg, 0.24 mmol) in methanol (1 mL) and the mixture was stirred at room temperature for 18 h. The solvent was evaporated under reduced pressure and the residue was triturated with ether. The solid was collected, suspended in aqueous sodium hydroxide (1M, 10 mL) and extracted with dichloromethane (2×20 mL). The combined organic fractions were washed with brine, dried (MgSO₄) and the solvent was evaporated under reduced pressure. The residue was purified by flash column chromatography on silica gel, eluting with CH₂Cl₂/MeOH/NH₃(Aq.) (40:8:1),to give the title compound as an off-white solid (38 mg, 30%). 1H NMR (250 MHz, CD₃OD) δ7.66 (1H, d, J 1.7 Hz), 7.60 (2H, d, J 8.5 Hz), 7.44 (3H, m), 7.32-7.15 (6H, m), 3.70-3.00 T1486 (4H, br m), 3.59 (3H, s), 3.08 (2H, t, J 7.2 Hz), 2.73 (2H, t, J 7.2 Hz), 2.61 (2H, s), and 1.60-0.80 (4H, br m). m/z (ES⁺) 520, 522 (M+1).

### EXAMPLE 68

### 1-{3-[5-Chloro-2-(4-Chlorophenyl)-1-Methyl-1H-Indol-3-yl]-1-Iminopropyl}-4-Cyclohexyl-4-Piperidinol

Prepared from 5-chloro-2-(4-chlorophenyl)-1-methyl-1H-indole-3-propanimidate hydrochloride (Description 51) and 4-cyclohexyl-4-piperidinol (Description 25), according to the method of Example 67. (360 MHz, CD₃OD) δ7.67 (1H, d, J 1.9 Hz), 7.61 (2H, d, J 8.6 Hz), 7.44 (3H, m), 7.23 (1H, dd, J 8.4, 1.9 Hz), 3.68 (1H, m), 3.58 (3H, s), 3.41-3.04 (5H, m), 2.76 (2H, t, J 7.2 Hz), 1.87-1.57 (5H, m), and 1.40-0.79 (10H, m). m/z (ES⁺) 512, 514 (M+1).

### EXAMPLE 69

### 1-{3- [5-Chloro-2-(4-Chlorophenyl)-1-Methyl-1H-Indol-3-yl]-1-Oxopropyl}-4-Fluoro-4-(Phenylmethyl)Piperidine

Prepared from 5-methyl-2-phenyl-1H-indole-3-propanoic acid (Description 5) or 5-chloro-2-(4-chlorophenyl)-1-methyl-1H-indole-3-propanoic acid (Description 11) and 4-fluoro-4-(phenylmethyl)piperidine (J.Med.Chem. 1999, 42, 2087-2104), according to the method of Example 26. ¹H NMR 360 MHz, CDCl₃) δ7.58 (1H, d, J 1.9 Hz), 7.47 (2H, d, J 8.5 Hz), 7.30-7.14 (9H, m), 4.44 (1H, m), 3.54 (3H, s), 3.40 (1H, m), 3.12 (1H, m), 2.98 (2H, m), 2.85-2.75 (3H, m), 2.48 (2H, t, J 7.8 Hz), and 1.78-1.10 (4H, m). m/z (ES⁺) 523, 525 (M+1).

### EXAMPLE 70

### 1-{3- [5-Chloro-2-(4-Chlorophenyl)-1-Methyl-1H-Indol-3-yl]Propyl}-4-{[(4-Fluorophenyl)Methyl]Sulfinyl}Piperidine

Prepared from 5-chloro-2-(4-chlorophenyl)-1-methyl-1H-indole-3-propanal (Description 23) and 4-[[(4-fluorophenyl)methyl]sulfinyl]piperidine (PCT Int. Appl. WO 96/04274. Chem. Abstr. 1996, 125, 58520) according to the method of Example 54. ¹H NMR (400 MHz, CDCl₃) δ7.59 (1H, d, J 1.5 Hz), 7.47 (2H, d, J 8.4 Hz), 7.31-7.18 (6H, m), 7.05 (2H, m), 3.96 (1H, d, J 13.2 Hz), 3.81 (1H, d, J 13.2 Hz), 3,54 (3H, s), 2.92 (1H, m), 2.84 (1H, m), 2.65 (2H, t, J 7.6 Hz), 2.46 (1H, m), 2.26 (2H, t, J 7.2 Hz), and 2.04-1.65 (8H, m).

The following compounds were prepared from 5-chloro-2-(4-chlorophenyl)-1-methyl-1H-indole-3-propanal (Description 23) according to the method of Example 54, substituting a suitable amine for 4-(phenylmethyl)-4-piperidinol.

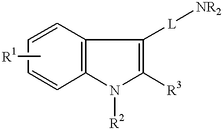

| | | | | | | | | m/z |
|---|---|---|---|---|---|---|---|---|
| | | | | | | | | (ES⁺) |
| | | | | | | | | (M + |
| Ex. | R¹ | R² | R³ | —L— | —NR₂ | Formula | M.W. | 1). |
| 71 | 5-Chloro | 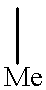 | 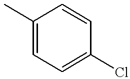 | 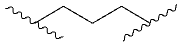 | 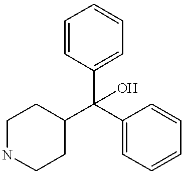 | C36H36Cl2N2O | 582 584 | 583 585 |
| 72 | 5-Chloro | 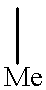 | 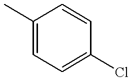 | 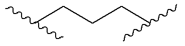 | 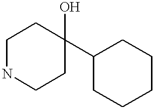 | C29H36Cl2N2O | 498 500 | 499 501 |
| 73 | 5-Chloro | 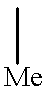 | 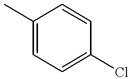 | 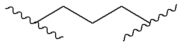 | 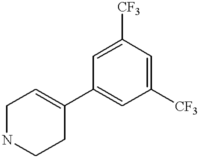 | C31H26Cl2F6N2 | 610 612 | 611 613 |
| 74¹ | 5-Chloro | 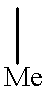 | 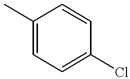 | 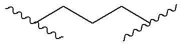 | 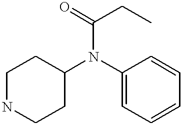 | C32H35Cl2N3O | 547 549 | 548 550 |
| 75 | 5-Chloro | 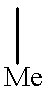 | 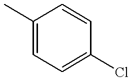 | 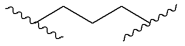 | 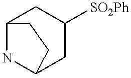 | C31H32Cl2N2O2S | 566 568 | 567 569 |
| 76 | 5-Chloro | 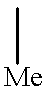 | 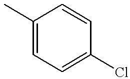 | 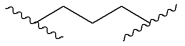 | 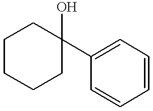 | C29H30Cl2N2O | 492 494 | 493 495 |

¹4-Cyclohexyl-4-piperidinol; Description 25.

### EXAMPLE 77

### (RS)-1-1-3-[{5-Chloro-2-(4-Chlorophenyl)-1-Methyl-1H-Indol-3-yl]-2-Hydroxypropyl}-4-(Phenylmethyl)-4-Piperidinol

Potassium carbonate (100 mg, 0.7 mmol) was added to a solution of (RS)-5-chloro-α-(chloromethyl)-2-(4-chlorophenyl)-1H-indole-3-ethanol (Description 53, 28 mg, 0.08 mmol) in methanol (2 mL) and the mixture was stirred at room temperature for 16 h. 4-(Phenylmethyl)-4-piperidinol (13 mg, 0.07 mmol) in methanol (1 mL) was added and the mixture was stirred at 60° C. for 20 h. The mixture was cooled and the solvent was evaporated under reduced pressure. Water was added and the mixture was extracted with ethyl acetate. The combined organic fractions were dried (Na₂SO₄) and the solvent was evaporated under reduced pressure. The residue was dissolved in methanol (1.5 mL) and poured onto an SCX cartridge (Varian Bond Elut; 10 mL/500 mg). The cartridge was washed with methanol (4×1.5 mL) and eluted with methanolic ammonia (2M, 3×1.5 mL). The solvent was evaporated under reduced pressure to give the title compound (10 mg, 24%). ¹H NMR (360 MHz, CDCl₃) δ7.64 (1H, d, J 1.8 Hz), 7.46 (2H, d, J 8.4 Hz), 7.38 (2H, d, J 8.4 Hz), 7.32-7.17 (7H, m), 3.91 (1H, m), 3.54 (3H, s), 2.93 (1H, m), 2.83-2.63 (2H, m), 2.73 (2H, s), 2.49 (2H, m), 2.29-2.13 (3H, m), and 1.75-1.46 (6H, m). m/z (ES⁺) 523, 525 (M+1).

### EXAMPLE 78

### 1-{3- [5-Chloro-2-(4-Chlorophenyl)-1-Methyl-1H-Indol-3-yl]-2-Oxopropyl}-4-(Phenylmethyl)-4-Piperidinol

1,1,1-Tris(acetyloxy)-1,1-dihydro-1,2-benziodoxol-3(1H)-one (50 mg, 0.12 mmol) was added to a solution of (RS)-1-{3-[5-chloro-2-(4-chlorophenyl)-1-methyl-1H-indol-3-yl]-2-hydroxypropyl}-4-(phenylmethyl)-4-piperidinol (Example 77, 24 mg, 0.04 mmol) in dichloromethane (2 mL) and the mixture was stirred at room temperature for 30 min. Further 1,1,1-tris(acetyloxy)-1,1-dihydro-1,2-benziodoxol-3(1H)-one (50 mg, 0.12 mmol) was added and the mixture was stirred at room temperature for 30 min. Further 1,1,1-tris(acetyloxy)-1,1-dihydro-1,2-benziodoxol-3(1H)-one (50 mg, 0.12 mmol) was added and the mixture was stirred at room temperature for 30 min. Saturated aqueous sodium carbonate (2 mL) and water (2 mL) were added slowly and the mixture was extracted with ethyl acetate (5 mL). The combined organic fractions were dried (MgSO₄) and the solvent was evaporated under reduced pressure. The residue was dissolved in methanol (1.5 mL) and poured onto an SCX cartridge (Varian Bond Elut; 10 mL/500 mg). The cartridge was washed with methanol (4×1.5 mL) and eluted with methanolic ammonia (2M, 3×1.5 mL). The solvent was evaporated under reduced pressure. The residue was purified by flash column chromatography on silica gel, eluting with CH₂Cl₂/MeOH/NH₃(Aq.) (97.5:2.5:0.25), then further purified by flash column chromatography on silica gel, eluting with isohexane/EtOAc (70:30 increasing to 40:60), to give the title compound (6 mg, 25%). ¹H NMR (250 MHz, CDCl₃) δ7.54 (1H, d, J 1.5 Hz), 7.47 (2H, d, J 8.5 Hz), 7.36-7.18 (9H, m), 3.74 (2H, s), 3.58 (3H, s), 3.14 (2H, s), 2.75 (2H, s), 2.53 (2H, m), 2.29 (2H, m), 1.76 (2H, m), 1.58 (1H br s), and 1.48 (2H, m). m/z (ES⁺) 521, 523 (M+1).

### EXAMPLE 79

### (RS)-1-{3- [5-Chloro-2-(4-Chlorophenyl)-1-Methyl-1H-Indol-3-yl]-3-[4-(Phenylmethyl)Piperidin-1-yl]Propan-2-ol

4-(Phenylmethyl)piperidine (22 mg, 0.12 mmol) was added to a solution of (RS)-5-chloro-2-(4-chlorophenyl)-1H-3-(oxiranylmethyl)indole (Description 54, 28 mg, 0.08 mmol) in acetonitrile (2 mL) and the mixture was stirred at 80° C. for 48 h. The mixture was cooled and the solvent was evaporated under reduced pressure. Butanenitrile (3 mL) was added and the mixture was stirred under reflux for 20 h. The mixture was cooled and methylisocyanate polysyrene HL resin, 200-400mesh, 2% DVB (Novabiochem, product no. 01-64-0169, 100 mg) was added. The mixture was stirred at room temperature for 1 h. The mixture was poured onto an SCX cartridge (Varian Bond Elut; 10 mL/500 mg). The cartridge was washed with methanol (4×1.5 mL) and eluted with methanolic ammonia (2M, 3×1.5 mL). The solvent was evaporated under reduced pressure to give the title compound (26 mg, 61%). ¹H NMR (250 MHz, CDCl₃) δ7.63 (1H, d, J 1.5 Hz), 7.46 (2H, d, J 8.5 Hz), 7.38 (2H, d, J 8.5 Hz), 7.29-7.10 (7H, m), 3.89 (1H, m), 3.54 (3H, s), 2.90-2.64 (4H, m), 2.50 (2H, d, J 6.7 Hz), 2.18 (3H, m), 1.77 (1H, m), and 1.64-1.12 (6H, m).

### EXAMPLE 80

### (RS)-1-{3- [5-Chloro-2-(4-Chlorophenyl)-1-Methyl-1H-Indol-3-yl]-2-Fluoropropyl}-4-(Phenylmethyl)-4-Piperidinol

Potassium carbonate (105 mg, 0.76 mmol) was added to a solution of (RS)-5-chloro-2-(4-chlorophenyl)-3-(3-chloro-2-fluoropropyl)-1H-indole (Description 55, 72 mg, 0.19 mmol) and 4-(phenylmethyl)-4-piperidinol (50 mg, 0.26 mmol) in 2-methylpropan-2-ol (3 mL) and the mixture was stirred under reflux for 20 h. Sodium iodide (80 mg, 0.5 mmol) was added and the mixture was stirred under reflux for 2 h. The mixture was cooled and the solvent was evaporated under reduced pressure. Butanenitrile (3 mL) was added and the mixture was stirred under reflux for 20 h. The mixture was cooled, water (5 mL) was added and the mixture was extracted with dichloromethane (10 mL). The organic fraction was treated with methylisocyanate polysyrene HL resin, 200-400mesh, 2% DVB (Novabiochem, product no. 01-64-0169, 200 mg) and the mixture was stirred at room temperature for 30 min. The mixture was poured onto an SCX cartridge (Varian Bond Elut; 10 mL/500 mg). The cartridge was washed with methanol (4×1.5 mL) and eluted with methanolic ammonia (2M, 3×1.5 mL). The residue was purified by flash column chromatography on silica gel, eluting with CH₂Cl₂/EtOAc (85:15), to give the title compound (10 mg, 10%). ¹H NMR (500 MHz, DMSO-d₆+CF₃CO₂H; 333K) δ7.72 (1H, s), 7.60 (2H, d, J 8.0 Hz), 7.48 (3H, m), 7.28 (2H, t, J 7.5 Hz), 7.21 (4H, m), 5.15 (1H, br d, J 49.0 Hz), 3.55 (3H, s), 3.30 (4H, m), 3.14 (2H, m), 2.97 (2H, m), 2.74 (2H, s), 1.76 (2H, m), and 1.59 (2H, m). m/z (ES⁺) 525, 527 (M+1).

What we claim is:

1. A compound of the formula (I): 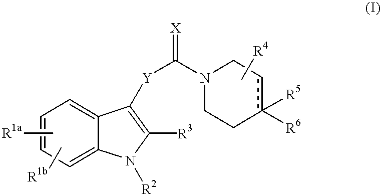
wherein
R^{1a} and R^{1b} each independently represent hydrogen, C₁₋₆alkyl, C₂₋₆alkenyl, C₁₋₆alkoxy, fluoroC₁₋₆alkyl, fluoroC₁₋₆alkoxy, halogen, cyano, NR^{a}R^{b}, SR^{a}, SOR^{a}, SO₂R^{a}, OSO₂R^{a}, NR^{a}COR^{b}, COR^{a}, CO₂R^{a}, CONR^{a}R^{b}, phenyl or heteroaryl, wherein said phenyl or heteroaryl group may be optionally substituted by one, two or three groups independently selected from halogen, C₁₋₆alkyl, C₁₋₆alkoxy, fluoroC₁₋₆alkyl, fluoroC₁₋₆alkoxy, NO₂, cyano, SR^{a}, SOR^{a}, SO₂R^{a}, COR^{a}, CO₂R^{a}, CONR^{a}R^{b}, C₂₋₆alkenyl, C₂₋₆alkynyl, C₁₋₄alkoxyC₁₋₄alkyl or —O(CH₂)₁₋₂O—;
R² represents hydrogen, C₁₋₆alkyl, fluoroC₁₋₆alkyl, (CH₂)ₘCOR^{a}, (CH₂)ₚCO₂R^{a}, (CH₂)ₚOH, (CH₂)ₘCONR^{a}R^{b}, (CH₂)ₘphenyl or SO₂C₁₋₆alkyl;
R³ represents phenyl, biphenyl, naphthyl or heteroaryl, wherein said phenyl, biphenyl, naphthyl or heteroaryl group may be optionally substituted by one, two or three groups independently selected from halogen, C₁₋₆alkyl, C₁₋₆alkoxy, fluoroC₁₋₆alkyl, fluoroC₁₋₆alkoxy, NO₂, cyano, SR^{a}, SOR^{a}, SO₂R^{a}, COR^{a}, CO₂R^{a}, CONR^{a}R^{b}, C₂₋₆alkenyl, C₂₋₆alkynyl, C₁₋₄alkoxyC₁₋₄alkyl or —O(CH₂)₁₋₂O—;
R⁴ represents hydrogen, C₁₋₆alkyl, carbonyl (═O), (CH₂)ₚphenyl or a C₁₋₂alkylene bridge across the piperidine ring;
R⁵ and R⁶ each independently represent hydrogen, halogen, C₁₋₆alkyl, C₃₋₇cycloalkyl, C₃₋₇cycloalkylC₁₋₄alkyl, C₂₋₆alkenyl, cyano, phenyl, naphthyl, fluorenyl, heteroaryl, (CH₂)pphenyl, (CH₂)pheteroaryl, CH(phenyl)₂, CH(C₁₋₆alkyl)(phenyl), (C₁₋₆alkyl)(phenyl)₂, CO(phenyl), C(OH)(phenyl)₂, C₂₋₄alkenyl(phenyl), (CH₂)ₘNR^{c}R^{d}, (CH₂)ₚCONR^{c}R^{d}, (CH₂)ₚNR^{a}COR^{b}, (CH₂)ₘCORC, (CH₂)ₘCO₂R^{c} or (CH₂)ₘOH wherein said phenyl, naphthyl, fluorenyl or heteroaryl groups may be optionally substituted by one, two or three groups independently selected from halogen, C₁₋₆alkyl, C₁₋₆alkoxy, fluoroC₁₋₆alkyl, fluoroC₁₋₆alkoxy, NO₂, cyano, SR^{a}, SOR^{a}, SO₂R^{a}, COR^{a}, CO₂R^{a}, CONR^{a}R^{b}, C₂₋₆alkenyl, C₂₋₆alkynyl, C₁₋₄alkoxyC₁₋₄alkyl or —O(CH₂)₁₋₂O—; or
R⁵ and R⁶ together are linked so as to form a 5- or 6-membered ring optionally substituted by ═O, ═S or a C₁₋₄alkyl or hydroxy group, and optionally containing a double bond, which ring may optionally contain in the ring one or two heteroatoms selected from O and S, or groups selected from NR^{c}, SO or SO_{2,} and to which ring there is either fused or attached a benzene or thiophene ring, which benzene or thiophene ring is optionally substituted by 1, 2 or 3 substituents selected from C₁₋₆alkyl, C₃₋₇cycloalkyl, C₃₋₇cycloalkylC₁₋₄alkyl, phenylC₁₋₄alkyl, trifluoromethyl, cyano, OR^{a}, SR^{a}, SOR^{a}, SO₂R^{a}, NR^{a}R^{b}, NR^{a}COR^{b}, NR^{a}CO₂R^{b}, NR^{a}SO₂R^{b}, COR^{a}, CO₂R^{a} or CONR^{a}R^{b}, wherein the phenyl moiety of a phenylC₁₋₄alkyl group may be substituted by C₁₋₆alkyl, C₁₋₆alkoxy, halogen or trifluoromethyl;
R^{a} and R^{b} each independently represent hydrogen, C₁₋₄alkyl, fluoroC₁₋₄alkyl or phenyl; or
the group —NR^{a}R^{b}may form a 5- or 6-membered ring optionally substituted by ═O, ═S or a C₁₋₄alkyl or hydroxy group, and optionally containing a double bond, which ring may optionally contain in the ring one or two heteroatoms selected from O and S, or groups selected from NRC, SO or SO₂;
R^{c} and R^{d} each independently represent hydrogen, C₁₋₄alkyl, fluoroC₁₋₄alkyl, C₂₋₄alkenyl, COR^{a}, SO₂R^{a}, phenyl or benzyl or R^{c} and R^{d}, together with the nitrogen atom to which they are attached, form a heteroaliphatic ring of 4 to 7 atoms, to which ring there may optionally be fused a benzene ring;
X represents an oxygen atom, a sulfur atom, two hydrogen atoms, ═NH or ═N(C₁₋₆alkyl);
Y is a straight or branched C₁₋₄alkylene chain optionally substituted by halogen, oxo or hydroxy; or
Y represents a straight or branched C₂₋₄alkenylene or C₂₋₄alkynylene chain;
the dotted line represents an optional double bond, with the proviso that when the double bond is present, R⁶ is absent;
m is zero or an integer from 1 to 4;
n is an integer from 1 to 4;
p is an integer from 1 to 4;
or a pharmaceutically acceptable salt thereof.

2. A compound as claimed in claim 1 wherein R^{1a} and R^{1b} each independently represent hydrogen, halogen, C₁₋₆alkyl, C₂₋₆alkenyl, fluoroC₁₋₆alkoxy, NR^{a}R^{b}, COR^{a}, CO₂R^{a}, or heteroaryl.

3. A compound as claimed in claim 1 wherein R² represents hydrogen, C₁₋₆alkyl, fluoroC₁₋₆alkyl, (CH₂)ₘCOR^{a}, (CH₂)ₚCOR^{a}, (CH₂)ₚOH or (CH₂)ₘphenyl.

4. A compound as claimed in claim 1 wherein R³ represents phenyl, biphenyl, naphthyl or heteroaryl wherein said phenyl group is optionally substituted by one or two groups selected from halogen, C₁₋₆alkyl, C₁₋₆alkoxy, trifluoroC₁₋₆alkyl, fluoroC₁₋₆alkoxy or C₂₋₆alkenyl.

5. A compound as claimed in claim 1 wherein R⁴ represents hydrogen, methyl, carbonyl, benzyl or a methylene bridge across the 2,5-positions on the piperazine ring.

6. A compound as claimed in claim 1 wherein R⁵ represents halogen, C₃₋₇cycloalkyl, C₃₋₇cycloalkylC₁₋₄alkyl, phenyl, heteroaryl, (CH₂)ₚphenyl, (CH₂)ₚheteroaryl, CH(phenyl)₂, CH(C₁₋₆alkyl)(phenyl), C(C₁₋₆alkyl)(phenyl)₂, CO(phenyl), C(OH)(phenyl)₂, or (CH₂)ₚNR^{c}R^{d}, wherein said phenyl or heteroaryl group is optionally substituted by one or two substituents selected from halogen, C₁₋₆alkyl, C₁₋₆alkoxy, fluoroC₁₋₆alkyl, fluoroC₁₋₆alkoxy, NO₂, cyano, SR^{a} or —O(CH₂)₁₋₂O—.

7. A compound as claimed in claim 1 wherein R⁶ represents hydrogen, fluorine, cyano, (CH₂)ₘNR^{c}R^{d}, (CH₂)ₚNR^{a}COR^{b}, (CH₂)ₘCO₂R^{c} or (CH₂)ₘOH, where R^{a}, R^{b}, R^{c} and R^{d} are as defined in claim 1.

8. A compound as claimed in claim 1 wherein R⁵ and R⁶ taken together form a 5- or 6-membered ring optionally substituted by ═O or a hydroxy group, and optionally containing a double bond, which ring optionally contains in the ring an oxygen or sulfur atom or 1 or 2 NH groups, and to which ring is either fused or attached a benzene ring, which benzene ring is optionally substituted by C₁₋₃alkyl or SO₂R^{a}, where R^{a} is as defined in claim 1.

9. A compound as claimed in claim 8 wherein R⁵ and R⁶ are so linked as to form a 5- or 6-membered ring in which said CR⁵R⁶ moiety is selected from:

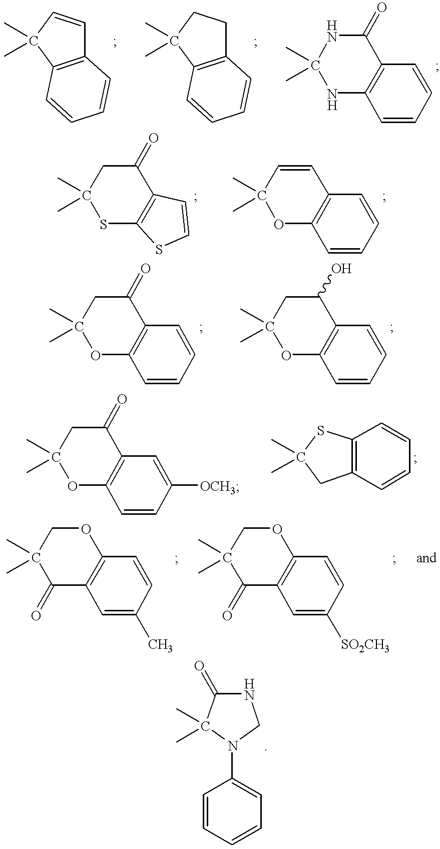

10. A compound as claimed in claim 1 wherein X represents an oxygen atom, two hydrogen atoms, or ═NH.

11. A compound as claimed in claim 1 wherein Y is —CH₂CH₂—, —CH₂CH(CH₃)—, —CH═CH— or —C≡C—.

12. A compound as claimed in claim 1 wherein X is two hydrogen atoms and Y is —CH₂CH₂—, —CH₂C(O)—, —CH₂CHOH— or —CH₂CHF—.

13. A compound of the formula (Ia) 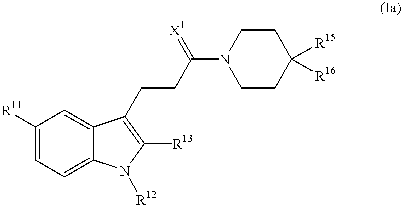
or a pharmaceutically acceptable salt thereof wherein
R¹¹ represents a chlorine or bromine atom or a methyl, vinyl, N-pyrrolidinyl, N-piperidinyl, N-morpholino, methoxycarbonyl, acetyl, 3-pyridyl or 2-furyl group;
R¹² represents a hydrogen atom or a methyl or acetyl group;
R¹³ represents 2-pyridyl, 3-pyridyl, unsubstituted phenyl, or phenyl substituted by a halogen atom;
R¹⁵ represents cyclohexyl, phenyl, 2-indolyl, CH₂phenyl, CH₂CH₂phenyl, CO(p-methoxyphenyl), C(OH)(phenyl)₂, NR^{c}R^{d} or CH₂NR^{c}R^{d} (where R^{c} and R^{d} each independently represent hydrogen, methyl, COCH₃, COCH₂CH₃, SO₂CH₃ or phenyl, or R^{c} and R^{d}, together with the nitrogen atom to which they are attached, form a piperidine ring) and wherein each phenyl group is optionally substituted by one or two substituents selected from fluorine, chlorine, bromine, methyl, methoxy, trifluoromethoxy or SO₂CH₃;
R¹⁶ represents hydrogen, fluorine, cyano, NR^{c}R^{d} (where R^{c} and R^{d} each independently represent hydrogen or methyl), NHCOCH₃, CH₂NHCOCH₃, CO₂H, CO₂CH₃, OH or CH₂OH; or
R¹⁵ and R¹⁶ together are so linked as to form a 5- or 6-membered ring optionally substituted by ═O, and optionally containing a double bond, which ring optionally contains in the ring an oxygen or sulfur atom or 1 or 2 NH groups, and to which ring is either fused or attached a benzene ring, which benzene ring is optionally substituted by methyl or SO₂CH₃; and
X¹ represents an oxygen atom or ═NH.

14. A compound as claimed in any preceding claim for use in therapy.

15. A pharmaceutical composition comprising a compound as claimed in claim 1, together with at least one pharmaceutically acceptable carrier or excipient.

16. A method for the treatment or prevention of physiological disorders associated with an excess of tachykinins, which method comprises administration to a patient in need thereof of a tachykinin reducing amount of a compound according to claim 1.

17. A method for the treatment or prevention of pain or inflammation, migraine, emesis, postherpetic neuralgia, depression or anxiety, which method comprises administration to a patient in need thereof of a therapeutically effective amount of a compound according to claim 1.
